SEQUENCE LISTING (20 sequences)

SEQ ID NO: 1 - protein, 606 aa, chicken
MPRRGAEGPLALLLAAAWLAQPLRGGYPGLNMFAVQTAQPDPCYDEHGLPRRCIPDFVNSAFGKEVKVSSTCGKPPSRYCVVTEKGEEQVRSCHLCNASDPKRAHPPSFLTDLNNPHNLTCWQSDSYVQYPHNVTLTLSLGKKFEVTYVSLQFCSPRPESMAIYKSMDYGKTWVPFQFYSTQCRKMYNKPSRAAITKQNEQEAICTDSHTDVRPLSGGLIAFSTLDGRPTAHDFDNSPVLQDWVTATDIKVTFSRLHTFGDENEDDSELARDSYFYAVSDLQVGGRCKCNGHASRCVRDRDDNLVCDCKHNTAGPECDRCKPFHYDRPWQRATAREANECVACNCNLHARRCRFNMELYKLSGRKSGGVCLNCRHNTAGRHCHYCKEGFYRDLSKPISHRKACKECDCHPVGAAGQTCNQTTGQCPCKDGVTGITCNRCAKGYQQSRSPIAPCIKIPAAPPPTAASSTEEPADCDSYCKASKGKLKINMKKYCKKDYAVQIHILKAEKNADWWKFTVNIISVYKQGSNRLRRGDQTLWVHAKDIACKCPKVKPMKKYLLLGSTEDSPDQSGIIADKSSLVIQWRDTWARRLRKFQQREKKGKCRKA

SEQ ID NO: 2 - protein, 603 aa, mouse
MMRAVWEALAALAAVACLVGAVRGPGLSMFAGQAAQPDPCSDENGHPRRCIPDFVNAAFGKDVRVSSTCGRPPARYCVVSERGEERVRSCHLCNSSDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYYYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGFYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKMNMKKYCRKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 3 - protein, 580 aa, mouse
MPTWLWGLLLTAGTLSAALSPGLPASADPCYDEAREPRSCIPGLVNAALGREVLASSTCGRSANRVCDSSDPQRAHSADLLTSAPGTASPLCWRSDLLQQAPFNVTLTVPLGKAFELVFVSLRFCSAPPTSVALLKSQDHGRSWVPLGFFSSSCTLDYGRLPAPADGPSGPGPEALCFPAPQAQPDGGGLLAFSVQDGSPQGLDLDNSPVLQDWVTATDIRIVLTRPAIQGDTRDGGVTVPYSYSATELQVGGRCKCNGHASRCLLDTHGHLVCDCQHGTEGPDCSRCKPFYCDRPWQRATGQEAHACLACSCNGHARRCRFNMELYRLSGRRSGGVCLNCRHNTAGRHCHYCREGFYRDPGRVLSDRRACRACDCHPVGAAGKTCNQTTGQCPCKDGVTGLTCNRCAPGFQQSRSPVAPCVKTPVPGPTEESSPVEPQDCESHCRPARGSYRISLKKFCRKDYAVQVAVGARGEARGSWTRFPVAVLAVFRSGEERARRGSSALWVPTLDAACGCPRLLPGRRYLLLGGGPGAAAGSTAGRGQGLSAARGSLVLPWRDAWTRRLRRLQRRERRGRCGTA

SEQ ID NO: 4 - protein, 581 aa, chicken
LRLLLTTSVLRLARAANPFVAQQTPPDPCYDESGAPRRCIPEFVNAAFGKEVQASSTCGKPPTRHCDASDPRRAHPPAYLTDLNTAANMTCWRSETLHHLPHNVTLTLSLGKKFEVVYVSLQFCSPRPESTAIFKSMDYGKTWVPYQYYSSQCRKIYGKPSKATVTKQNEQEALCTDGLTDLYPLTGGLIAFSTLDGRPSAQDFDSSPVLQDWVTATDIRVVFSRPHLFRELGGREAGEEDGGAGATPYYYSVGELQVGGRCKCNGHASRCVKDKEQKLVCDCKHNTEGPECDRCKPFHYDRPWQRASAREANECLACNCNLHARRCRFNMELYKLSGRKSGGVCLNCRHNTAGRHCHYCKEGFYRDLSKSITDRKACKACDCHPVGAAGKTCNQTTGQCPCKDGVTGLTCNRCAKGFQQSRSPVAPCIKIPAINPTSLVTSTEAPADCDSYCKPAKGNYKINMKKYCKKDYVVQVNILEMETVANWAKFTINILSVYKCRDERVKRGDNFLWIHLKDLSCKCPKIQISKKYLVMGISENSTDRPGLMADKNSLVIQWRDAWTRRLRKLQRREKKGKCVKP

SEQ ID NO: 5 - protein, 627 aa, mouse
MGSCARLLLLWGCSAVAAGLNGVAGANSRCEKACNPRMGNLALGRKLRADTMCGQNATELFCFYSENADLTCRQPKCDKCNAAHSHLAHPPSAMADSSFRFPRTWWQSAEDVHREKIQLDLEAEFYFTHLIMVFKSPRPAAMVLDRSQDFGKTWKPYKYFATNCSATFGLEDDVVKKGAICTSRYSNPFPCTGGEVIFRALSPPYDIENPYSAKVQEQLKITNLRVRLLKRQSCPCINDLNAKPHHFMHYAVYDFIVKGSCFCNGHADQCLPVEGFRPIKAPGAFHVVHGRCMCKHNTAGSHCQHCAPLYNDRPWEAADGRTGAPNECRTCKCNGHADTCHFDVNVWEASGNRSGGVCNNCQHNTEGQHCQRCKPGFYRDLRRPFSAPDACKACSCHPVGSAILPFSSVTFCDPSNGDCPCKPGVAGPHCDRCMVGYWGFGDYGCRPCDCAGSCDPLTGDCISSNADVDWYHEVPTFHSMHNKSEPSWEWEDEQGFSALRHSGKCECKEQVLGNPKAFCGMKYSYVLKIKILSAHDKGSHAEVNVKIKKVLKSTKLKILRGKRTLYPESWTNRGCTCPILNPGLEYLVAGHEDVRTGKLIVNMKSFVQHWKPALGRRVMHILKRDCV

SEQ ID NO: 6 - protein, 604 aa, human
MMRAVWEALAALAAVACLVGAVRGGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKINMKKYCKKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 7 - protein, 580 aa, human
MPGWPWGLLLTAGTLFAALSPGPPAPADPCHDEGGAPRGCVPGLVNAALGREVLASSTCGRPATRACDASDPRRAHSPALLTSPGGTASPLCWRSESLPRAPLNVTLTVPLGKAFELVFVSLRFCSAPPASVALLKSQDHGRSWAPLGFFSSHCDLDYGRLPAPANGPAGPGPEALCFPAPLAQPDGSGLLAFSMQDSSPPGLDLDSSPVLQDWVTATDVRVVLTRPSTAGDPRDMEAVVPYSYAATDLQVGGRCKCNGHASRCLLDTQGHLICDCRHGTEGPDCGRCKPFYCDRPWQRATARESHACLACSCNGHARRCRFNMELYRLSGRRSGGVCLNCRHNTAGRHCHYCREGFYRDPGRALSDRRACRACDCHPVGAAGKTCNQTTGQCPCKDGVTGLTCNRCAPGFQQSRSPVAPCVKTPIPGPTEDSSPVQPQDCDSHCKPARGSYRISLKKFCKKDYAVQVAVGARGEARGAWTRFPVAVLAVFRSGEERARRGSSALWVPAGDAACGCPRLLPGRRYLLLGGGPGAAAGGAGGRGPGLIAARGSLVLPWRDAWTRRLRRLQRRERRGRCSAA

SEQ ID NO: 8 - protein, 504 aa, human
MGSCARLLLLWGCTVVAAGLSGVAGVSSRCEKACNPRMGNLALGRKLWADTTCGQNATELYCFYSENTDLTCRQPKCDKCNAAYPHLAHLPSAMADSSFRFPRTWWQSAEDVHREKIQLDLEAEFYFTHLIVMFKSPRPAAMVLDRSQDFGKTWKPYKYFATNCSATFGLEDDVVKKGAICTSKYSSPFPCTGGEVIFKALSPPYDTENPYSAKVQEQLKITNLRVQLLKRQSCPCQRNDLNEEPQHFTHYAIYDFIVKGSCFCNGHADQCIPVHGFRPVKAPGTFHMVHGKCMCKHNTAGSHCQHCAPLYNDRPWEAADGKTGAPNECRACKCNGHADTCHFDVNVWEASGNRSGGVCDDCQHNTEGQYCQRCKPGFYRDLRRPFSAPDACKPCSCHPVGSAVLPANSVTFCDPSNGDCPCKPGVAGRRCDRCMVGYWGFGDYGCRPCDCAGSCDPITGDCISSHTDIDWYHEVPDFRPVHNKSEPALGVGGCAGVFCTSTLR

SEQ ID NO: 9 - protein, 429 aa, chicken
GYPGLNMFAVQTAQPDPCYDEHGLPRRCIPDFVNSAFGKEVKVSSTCGKPPSRYCVVTEKGEEQVRSCHLCNASDPKRAHPPSFLTDLNNPHNLTCWQSDSYVQYPHNVTLTLSLGKKFEVTYVSLQFCSPRPESMAIYKSMDYGKTWVPFQFYSTQCRKMYNKPSRAAITKQNEQEAICTDSHTDVRPLSGGLIAFSTLDGRPTAHDFDNSPVLQDWVTATDIKVTFSRLHTFGDENEDDSELARDSYFYAVSDLQVGGRCKCNGHASRCVRDRDDNLVCDCKHNTAGPECDRCKPFHYDRPWQRATAREANECVACNCNLHARRCRFNMELYKLSGRKSGGVCLNCRHNTAGRHCHYCKEGFYRDLSKPISHRKACKECDCHPVGAAGQTCNQTTGQCPCKDGVTGITCNRCAKGYQQSRSPIAPCI

SEQ ID NO: 10 - protein, 429 aa, human
GGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCI

SEQ ID NO: 11 - protein, 260 aa, chicken
GYPGLNMFAVQTAQPDPCYDEHGLPRRCIPDFVNSAFGKEVKVSSTCGKPPSRYCVVTEKGEEQVRSCHLCNASDPKRAHPPSFLTDLNNPHNLTCWQSDSYVQYPHNVTLTLSLGKKFEVTYVSLQFCSPRPESMAIYKSMDYGKTWVPFQFYSTQCRKMYNKPSRAAITKQNEQEAICTDSHTDVRPLSGGLIAFSTLDGRPTAHDFDNSPVLQDWVTATDIKVTFSRLHTFGDENEDDSELARDSYFYAVSDLQVGG

SEQ ID NO: 12 - protein, 260 aa, human
GGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGG

SEQ ID NO: 13 - protein, 20 aa, mouse
KPFHYDRPWQRATAREANEC

SEQ ID NO: 14 - protein, 14 aa, mouse
IYKSMDYGRTWVPF

SEQ ID NO: 15 - protein, 18 aa, mouse
RFNMELYKLSGRKSGGVC

SEQ ID NO: 16 - protein, 10 aa, human
ITKQNEQEAV

SEQ ID NO: 17 - protein, 8 aa, human
TDSHTDMR

SEQ ID NO: 18 - protein, 21 aa, human
SAHDFDNSPVLQDWVTATDIR

SEQ ID NO: 19 - protein, 18 aa, human
KEGYYRDMGKPITHRKAC

SEQ ID NO: 20 - protein, 14 aa, human
AKGYQQSRSPIAPC

(19) United States
(12) Patent Application Publication
(10) Pub. No.: US 2012/0178690 A1
(43) Pub. Date: Jul. 12, 2012

(54) NOVEL NETRIN DERIVATIVES AND USES THEREOF

(75) Inventor: Timothy E. Kennedy, Montreal (CA)

(73) Assignee: The Royal Institution for the Advancement of Learning/McGill University, Montreal, QC (CA)

(21) Appl. No.: 13/377,418

(22) PCT Filed: Jun. 9, 2010
(86) PCT No.: PCT/CA2010/000900

§ 371 Date: Mar. 27, 2012

Related U.S. Application Data
(60) Provisional application No. 61/185,326, filed on Jun. 9, 2009.

(51) Int. Cl. A61K 38/17; C12Q 1/68; G01N 33/574; A61P 35/00; C07K 14/47; C07H 21/04

(52) U.S. Cl. 514/13.3; 514/19.3; 530/350; 536/23.5; 435/7.23; 435/6.12

(57) ABSTRACT

Netrin proteins and their receptors regulate cell and axon migration, and are implicated in tissue morphogenesis, tumorigenesis and angiogenesis. Deregulation of mechanisms that control cell motility plays a key role in tumor progression by promoting tumor cell dissemination. Unwanted neovascularization also contributes to tumor progression and metastasis and to ocular diseases which are a leading cause of blindness. Here, we describe novel netrin-derived polypeptides and fragments or derivatives thereof that selectively inhibit cell growth, migration or branching. Methods and compositions for the treatment and prevention of conditions involving cell migration or neovascularization, such as cancer and ocular disease, are also provided.

### FIELD OF THE INVENTION

The present invention relates to a novel method for controlling cell migration. Specifically, novel derivatives of netrin and compounds derived therefrom which have been found to function selectively as inhibitors of cell growth and/or migration are disclosed herein. The netrin derivatives and related compounds of the present invention lack the capacity of full length netrin to also promote cell migration and thus permit a novel approach to restrain cell growth, migration, and branching, which may be of use in the treatment of disease involving cell movement, metastasis or neovascularization, such as certain cancers and ocular disorders.

### BACKGROUND OF THE INVENTION

Netrins are a family of secreted, extracellular matrix proteins that direct cell and axon migration during neural development. They are bifunctional, acting as either chemoattractants or chemorepellents for different cell types (reviewed by Manitt and Kennedy, 2002, Prog. Brain Res. 137:425-442). Five netrins have been identified in mammals, netrin-1, 3, 4, G1 and G2. All are about 75 kDa in size and share amino terminal sequence homology with laminins, which are large extracellular matrix proteins. Netrins are composed of three domains, termed the VI, V and C domains.

Receptors for netrin-1 include Deleted in Colorectal Cancer (DCC) and the UNC5 homologue family (Manitt and Kennedy, 2002; see also FIGS. 1 and 7). DCC is a single pass transmembrane Ig superfamily member that is required for the attractant response to netrin-1. UNC5 homologues are single pass transmembrane proteins required for repellent responses to netrin-1. Four UNC5 family members, UNC5A, B. C, and D, have been identified in mammals. Many cells express both UNC5 homologues and DCC and these cells appear to have the capacity to respond to netrin as either an attractant or repellent. Netrin-1 and netrin receptors are expressed in many adult tissues, but their function in the adult remains unknown. Netrin-1 is widely expressed by neurons and glia in the adult CNS (Manitt, C., et al., 2001, J. Neurosci. 21:3911-3922), and reduced expression has been documented in brain tumors, including glioblastoma (Meyerhardt. J. A., et al., 1999, Cell Growth Differ. 10:35-42), which is one of the most lethal forms of brain tumor.

Cell migration is essential for normal embryonic development, wound healing, and immunity, but it can be devastating in disease states, such as tumor invasion and metastasis. Deregulation of mechanisms that control cell motility plays a key role in tumor progression by promoting tumor cell dissemination. For example, a major problem for the treatment of glioblastoma is that tumor cells escape surgical removal by migrating away from the site of initial tumor formation. These migrating cells seed new tumor foci in other brain regions, leading the tumor to recur. Many tumor cells acquire the ability to migrate far from their place of origin, in some cases metastasizing and invading surrounding tissue. Identifying means to inhibit glioblastoma dissemination within the CNS is thus an urgent unmet medical need (Henson, J. W., 2006. Arch. Neurol. 63:337-341).

There is a need for inhibitors of cell migration that can be used for example to treat cancer and tumor metastasis. There is also a need for inhibitors of neovascularization that can be used to block tumor growth or in the treatment of ocular disorders such as age-related macular degeneration and retinal disease.

### SUMMARY OF THE INVENTION

We demonstrate herein that netrin-1 inhibits human glioblastoma cell migration and report the surprising finding that a recombinant fragment of netrin-1 comprising the VI-V domains (the VI-V peptide, ˜45 kDa) selectively inhibits cell migration, including glioblastoma migration, without evoking a chemoattractant response or promoting migration. We have applied this peptide to human glioblastoma cell lines and demonstrated that application of VI-V peptide inhibits human glioblastoma cell migration without evoking a chemoattractant response or promoting migration. We have further reduced the size of the netrin-1 peptide, demonstrating that recombinant netrin-1 domain VI, fused to a human Fc protein, inhibits glial precursor cell migration at least as effectively as does full length netrin-1.

The present invention relates therefore to novel derivatives of netrin and compounds derived therefrom which can function selectively as inhibitors of cell growth, migration or branching. Our findings indicate a role for netrin in regulating cell motility and show that the netrin derivatives and related compounds disclosed herein can be used, for example, to inhibit tumor cell migration and dispersion. Specifically, methods and compositions relating to novel netrin-derived polypeptides capable of selectively inhibiting cell growth, migration or branching are described. The agents and compositions described herein also find use as therapeutic, prophylactic and diagnostic agents.

In accordance with the present invention, there is provided a method of inhibiting tumor cell migration in a subject, the method comprising contacting a tumor cell undergoing or likely to undergo movement with a netrin polypeptide in an amount effective to decrease migration of the tumor cell, thereby modulating cell migration in the subject. In an aspect, the tumor cell is a glioblastoma cell. In another aspect, the tumor cell may be a colorectal, breast or pancreatic tumor cell. The netrin polypeptide may be derived from netrin-1, netrin-2, netrin-3, netrin-4, netrin-G1, netrin-G2, recombinant netrin-1, recombinant netrin-2, recombinant netrin-3, recombinant-netrin-4, recombinant-netrin-G1, recombinant-netrin-G2, or variants, homologues, fragments or functional derivatives thereof. In an aspect, the polypeptide is the VI-V domain of netrin, the VI domain of netrin, or a fragment thereof. In another aspect, the polypeptide is derived from a vertebrate netrin, in particular from human netrin. In yet another aspect, the subject is a human.

There is further provided herein a method of inhibiting tumor cell migration in a subject, the method comprising contacting a tumor cell undergoing or likely to undergo movement with a netrin polypeptide and a laminin polypeptide in an amount effective to decrease migration of the tumor cell, thereby modulating cell migration in the subject. There is also provided herein a kit for modulating tumor cell migration in a subject, the kit comprising a netrin polypeptide that decreases tumor cell migration, optionally a laminin peptide, and instructions for using the netrin polypeptide to modulate tumor cell migration in the subject.

In another aspect, there is provided herein a method of promoting the maturation of focal complexes (FCs) into focal adhesions (FAs) to restrain tumor cell migration in a subject, the method comprising contacting a tumor cell undergoing or likely to undergo movement with a netrin polypeptide in an amount effective to promote the maturation of FCs into FAs in the subject, thereby restraining cell migration.

In accordance with another aspect of the present invention, there is provided herein an isolated polypeptide comprising the sequence of the VI-V domain of netrin or a fragment, analog or modification thereof, wherein the fragment, analog or modification selectively inhibits cell growth or migration, and lacks the capacity of full-length netrin to also promote cell migration. In an aspect, the isolated polypeptide is derived from a vertebrate netrin, for example human netrin-1, human netrin-3 or human netrin-4. In another aspect, the isolated polypeptide may be one of the polypeptides set forth in SEQ ID NOs: 1 to 20. Isolated polynucleotides encoding the polypeptides of the invention are also encompassed herein.

In another aspect, pharmaceutical compositions comprising the polypeptides of the invention and a pharmaceutically acceptable carrier are provided herein, as are kits containing the polypeptides of the invention and instructions for use.

In a further aspect, there is provided herein a method of treating or preventing cancer in a subject in need thereof, comprising administering a therapeutically effective amount of a subject polypeptide or composition of the invention to the subject, such that cancer is treated or prevented in the subject. In an aspect, tumor cell migration is inhibited in the subject. In another aspect, the maturation of focal complexes into focal adhesions is inhibited in the subject. In yet another aspect, neovascularization is inhibited in the subject. In a further aspect, a tumor cell undergoing or likely to undergo movement is contacted with the polypeptide. In one embodiment, the cancer may be, for example, colorectal cancer, glioblastoma, pancreatic cancer or breast cancer. Metastasis may be inhibited in the subject.

In a yet further aspect, there is provided a method of treating or preventing an ocular disease in a subject in need thereof, comprising administering a therapeutically effective amount of a netrin-derived polypeptide or compound derived therefrom, or a pharmaceutical composition, as described herein, to the subject, such that ocular disease is prevented or treated in the subject. In an aspect, the disease is associated with neovascularization. In another aspect, the disease is age-related macular degeneration, diabetic retinopathy, or retinitis pigmentosa (RP). In yet another aspect, neovascularization is inhibited in the subject. In a yet further aspect, cell growth, migration or branching is inhibited in the subject.

In another aspect, a method of treating or preventing unwanted neovascularization in a subject in need thereof, comprising administering a therapeutically effective amount of a polypeptide or composition as described herein to a subject is provided. In one aspect, the subject has an ocular disease or cancer. In another aspect, the disease is colorectal cancer, glioblastoma, age-related macular degeneration, diabetic retinopathy, or retinitis pigmentosa (RP). Also provided herein is a method of inhibiting cell migration in a subject in need thereof, comprising administering a therapeutically effective amount of a polypeptide or composition of the invention to the subject. The subject may have, for example, an ocular disease or cancer, or the disease may be colorectal cancer, glioblastoma, age-related macular degeneration, diabetic retinopathy, or retinitis pigmentosa (RP).

Further in accordance with the present invention, there is provided herein a method for diagnosis or prognosis of multiple sclerosis in a subject in need thereof, comprising determining whether the VI-V domain of netrin, or a proteolytic fragment thereof, is present in CSF, in blood, or in a lesion in the subject.

### BRIEF DESCRIPTION OF THE FIGURES

Having thus generally described the nature of the invention, reference will now be made to the accompanying drawings, showing by way of illustration, a preferred embodiment thereof.

FIG. 1: Conserved structure of netrins and their receptors. (A) All netrins contain conserved amino terminal domains V and VI related to the amino terminal domains of laminins. Domain V contains cysteine-rich epidermal growth factor (EGF) repeats. Domain C in secreted netrins contains several positively charged, basic residues. (B) DCC and Unc5 homologues are receptors for netrin-1 to -4.

FIG. 2: Netrin does not affect U87 or U373 survival or proliferation in vitro. (A) Cell viability was assessed by labeling F-actin with Alexa 488-conjugated phalloidin, nuclei with Hoechst, and counting. Addition of netrin-1, laminin-1, or both did not affect U87 cell viability. Neither 25 μg/ml netrin function blocking antibody (Net_{FB}) nor 10 μg/ml DCC_{FB}, an antibody that blocks DCC function, affected cell number. Cell number did not change following addition of 100 ng/ml netrin-1 or 25 μg/ml Net_{FB} (16 hr assay). (B) To further assess apoptotic cell death under the conditions in panel A. cell lysates were analyzed by immunoblot for the active (cleaved) form of caspase-3, A 17 kDa caspase-3 band (black arrowhead) was only observed in lysates exposed to staurosporine, a potent inducer of apoptosis. The white arrowhead indicates a nonspecific 15 kDa immunoreactive band. (C) To determine if netrin acts as a ‘dependence receptor’ ligand, cells were treated with antibodies blocking either DCC or netrin function for 48 hours. As in panel B, only staurosporine treatment promoted cell death. Ctrl C control; Lam L laminin-1; Net N netrin-1; Net_{fb} Nₜₕ netrin function-blocking antibody; DCC_{fb} D_{fb} DCC function-blocking antibody; LN laminin-1 and netrin-1; ND_{fb} Netrin-1 and DCC_{fb}; LND_{fb} Laminin-1 netrin-1 and DCC_{fb}; R pre-immune rabbit IgG; St staurosporine.

FIG. 3: Glioblastoma cell lines express netrins and their receptors: Autocrine netrin inhibits U87 and U373 cell migration. (A) Western blot analysis of cell lysates or conditioned media from astrocytes (Ast), U343, U373, and U87 cells with molecular mass markers (kDa) as indicated to the left of each blot detect full-length netrin protein (˜75 kDa) in medium conditioned by each glioma cell line or by astrocytes. A band corresponding to full-length DCC protein (˜185 kDa) was detected in whole cell lysates of astrocytes (Ast) and U87 cells, but not U343 and U373 cells. This blot was overexposed to reveal DCC in astrocytes and its absence in U343 and U373 cells. U343 and U373 cells transfected with pDCC-GFP express DCC-GFP chimeric protein, which migrates at a slightly higher molecular weight than endogenous DCC (middle panel). A ˜190 kDa band, the molecular weight of the DCC paralogue neogenin was detected in lysates of all three cell lines (bottom panel, 30 μg protein/lane). (B) RT-PCR analysis of U87, U343 and U373 cell total RNA. (C) Transfilter microchemotaxis assays of U87, U343, and U373 motility. (D) U87 cell migration increased when 25 μg/ml netrin function-blocking antibody (Net_{FB}) was added to the top and bottom compartments, relative to medium alone (Control), or control antibody (Control IgG). 10 μg/ml DCC_{FB} did not increase migration. (F) Netrin function-blocking antibody (Net_{FB}) also significantly increased U373 cell migration, but had no effect on U343 cell migration (E). (G) Schematic of microchemotaxis assay. Number of cells migrated is per 10× objective field. Duration of microchemotaxis assays was 16 hrs. * p<0.05 vs. control.

FIG. 4: Netrin and netrin receptors are found in focal adhesions but not focal complexes. Three human glioblastoma cell lines U87. U343, and U373, were labeled with antibodies against paxillin (green) and netrin, DCC, and unc5 homologues (red; all panels except S-U) or zyxin (green) and DCC (red; S-U), and lamellipodia imaged. In U87 cells, small, paxillin-positive FCs localized at the lamellipodial edge were not netrin-positive (black arrowhead). Netrin immunoreactivity co-localizes with larger paxillin-positive structures located away from the lamellipodial edge (white arrowhead), consistent with FAs (A-C). UNC5 homologue (G-I) and DCC (M-O) immunoreactivity were similarly localized to FAs in U87 cells. DCC immunoreactivity co-localized with zyxin-positive FAs (S-U). Similarly, in U343 and U373 cells, netrin (D-F, P-R) and UNC5 homologue (J-L. V-X) co-localize with FAs, but not FCs. Confocal microscopy, 100× objective, scale bar=2 μm.

FIG. 5: Disrupting netrin function increases the number of focal complexes and reduces the number of focal adhesions in U87 and U373 lamellipodia. FCs in lamellipodia were identified and quantified by subtracting zyxin immunoreactivity from paxillin immunoreactivity, revealing localization of paxillin without zyxin. FAs in cell lamellipodia were identified and quantified by generating images of paxillin and zyxin co-localization and determining the density of paxillin+/zyxin+foci. 25 μg/ml control rabbit IgG (Rb IgG), 100 ng/ml netrin-1 or 10 μg/ml DCC_{FB} resulted in no change in FC or FA density relative to control medium. 25 μg/ml Net_{FB} significantly increased the density of FCs and decreased FA density. * p<0.05 vs. control.

FIG. 6: VI-V netrin-1 peptide selectively inhibits cell migration. (A)

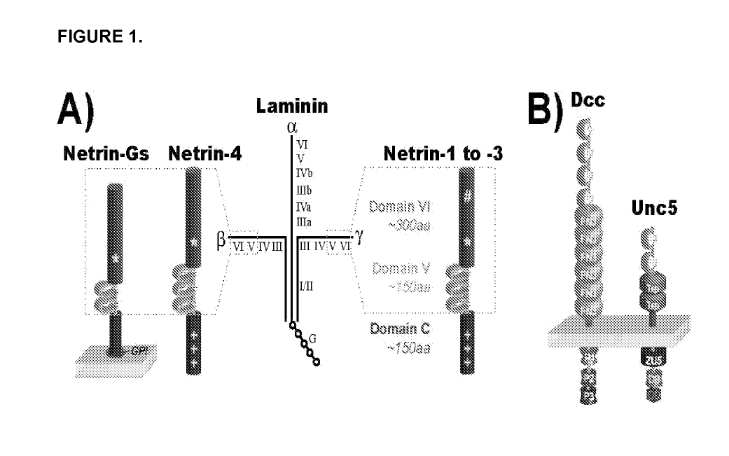

Schematic illustrating full-length netrin-1 protein and netrin-1 VI-V peptide. (B) The migration of oligodendroctye precursors (OPCs) isolated from newborn rat brain is inhibited in the transfilter microchemotaxis assay by 100 ng/ml netrin-1 VI-V peptide in the bottom chamber. 16 hr assay (cells isolated and assayed as described in Jarjour, A. A., et al., 2003, J. Neurosci. 23:3735-3744). (C) Dose response analysis of newborn rat brain OPCs migrating with netrin-1 VI-V peptide in the bottom chamber. Maximal levels of inhibition at ˜25 ng/ml. (D) The migration of human U373 glioblastoma cells in the transfilter migration assay is inhibited by 100 ng/ml netrin-1 VI-V peptide placed in either bottom well alone (VI-V B), or uniformly in the top and bottom wells VI-V TB). Net B: 100 ng/ml full-length netrin-1 in bottom well. Netrin-1 VI-V peptide inhibits U373 migration in the presence of full-length netrin-1 (netB+VI-V TB). (E) Collagen gel outgrowth assays demonstrate that embryonic rat spinal commissural axons respond to a gradient of netrin-1 as a chemoattractant (Net100, 100 ng/ml full length netrin-1). Application of 100 ng/ml netrin-1 VI-V does not evoke axon outgrowth (Net VI-V 100), and notably, netrin-1 VI-V peptide antagonizes the capacity of full length netrin-1 to promote outgrowth.

FIG. 7: Schematic illustrating the predicted structure of domain V of human netrin-1. Notably, loss of sub-domain V-2 in C. elegans causes loss of chemorepellent activity, while maintaining chemoattractant activity of the nematode netrin UNC-6 (Wadsworth, W. G., et al., 1996. Neuron 16:35-46). This mutation phenocopies loss of function of UNC5, the repellent receptor for netrins.

FIG. 8: Netrin-1 is a chemoattractant for DCC-expressing glioblastoma cells. (A,B) Addition of 100 ng/ml netrin-1 to the bottom compartment (NB) of the transfilter microchemotaxis assay significantly increased U87 cell migration compared to control (medium alone). NB: netrin-1 bottom. NTB: netrin-1 top and bottom. NB DCC_{FB}: netrin-1 bottom. DCC function-blocking antibody. Similar results were obtained in assays lasting (A) 16 hours and (B) 48 hours. (C) A gradient of netrin-1 had no effect on the migration of U343 or U373 cells. (D) U343 cells transfected with a DCC expression construct (U343D control), reduced their rate of migration relative to the parental line (U343P). Increased migration of DCC-transfected U343 cells was evoked by a netrin-1 gradient (U343D NB), but not uniform netrin-1 (NTB). DCC_{FB} blocked this response (U343D DCC_{FB}). (F) Transfection of U373 cells with DCC produced responses similar to U343 cells, which mimic those seen in DCC expressing U87 cells. Number of cells migrated is per 10× objective field. 16 hr assays in all panels except (B). * p<0.05 vs. control (A,B), U343P (D) or U373P (E). §p<0.05 vs. U343D Control (E) or U373D Control (F).

FIG. 9: U87 attraction to netrin is converted to repulsion by laminin-1. (A) U87 migration in the microchemotaxis assay challenged with an ascending gradient of laminin-1 (LB) increased relative to control (C). A uniform distribution of laminin-1 (LTB) does not increase U87 migration. An ascending gradient of netrin-1 and uniform laminin-1 (LTBNB), or uniform distributions of both netrin-1 and laminin-1 (LTBNTB), results in reduced U87 migration. Challenging cells with a descending gradient of netrin-1 with a uniform distribution of laminin-1 (LTBNT), evoked increased migration relative to control. Addition of DCC_{FB} to both the top and bottom compartments in the presence of a uniform distribution of laminin-1 and an ascending gradient of netrin-1 (LTBNB DCC_{FB}) or of uniform distributions of both netrin-1 and laminin-1 (LTBNTB DCC_{FB}) blocked the decrease in migration observed. (B) Schematic depicting migratory responses of U87 cells in (A). Migration assayed after 48 hrs. * p<0.05 vs. control.

FIG. 10: Model of tumor-suppressor activity of netrin. Model of tumor suppression by netrin in vivo. Upon encountering a laminin-1-rich basal lamina surrounding a blood vessel, glioblastoma cells expressing both netrin-1 and netrin receptors (A, left) are inhibited from migrating along the vessel by the combination of netrin-1 and laminin-1. Disruption of netrin signaling allows the cell to respond to laminin-1 as a permissive substrate, and migrate along the surface of blood vessels leading to tumor dissemination in the CNS. (B) We hypothesize that netrin may restrain cell motility and thereby contribute to maintaining the integrity of epithelial or endothelial cell layers in non-neural tissues.

FIG. 11: Peptide sequences. Sequences of the full-length chick netrin-1 protein, the processed VI-V domain of netrin, and the VI domain of netrin are shown.

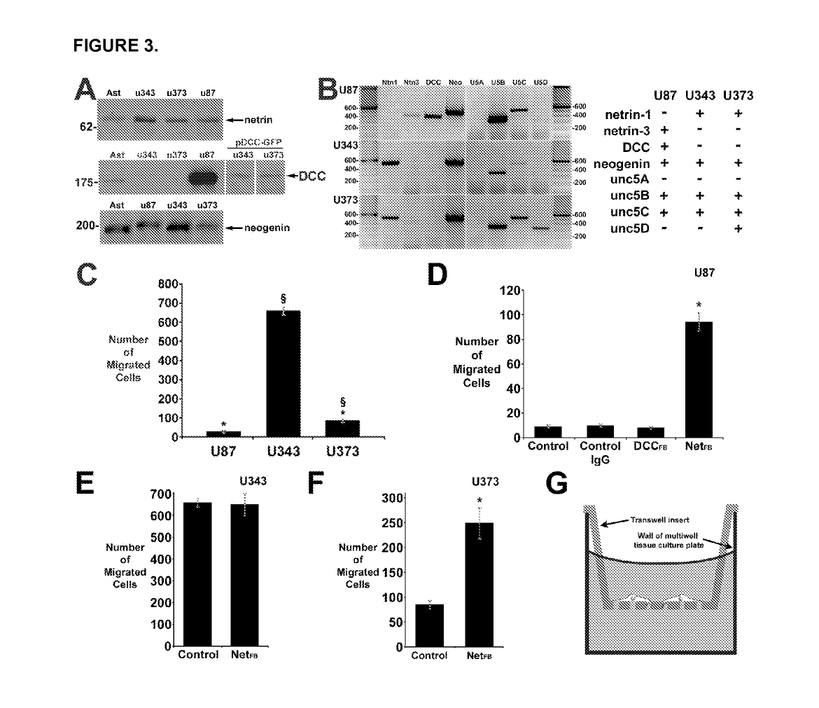

FIG. 12: Netrin-1 domain VI is sufficient to repel glial precursor cell migration. The histograms illustrate the migration of glial precursor cells (oligodendrocyte precursor cells isolated from newborn rat brain) in response to either full length purified recombinant netrin-1, or purified recombinant protein chimera composed of netrin-1 domain VI linked to a human antibody Fc domain. Migration was tested in a Boyden chamber as illustrated in FIG. 3C. Cells were plated on the top side of a porous membrane. In the control condition, the count corresponds to the number of cells that spontaneously migrated from the top side of the membrane to the bottom side of the membrane. In the netrin bottom (100 ug/ml) condition, full length netrin-1 protein placed in the bottom portion of the chamber repelled the migration of glial precursor cells across the membrane, resulting in fewer cells migrating than control. The leftmost 3 histogram bars illustrate the number of cells migrating in response to netrin-1 domain VI-Fc chimera placed in the bottom chamber. A dose response was carried out, placing either 100 ug/ml, 200 ug/ml, or 300 ug/ml in the bottom chamber, as indicated. The reduced number of cells migrating across the membrane indicates that netrin-1 domain VI is sufficient to repel glial precursor cell migration. Four independent replicates of each condition were used: n=4. Statistical comparison utilized an ANOVA test with a Tukey HSD correction; * indicates P<0.001 compared to control.

FIG. 13: Expression of netrin-1 and netrin receptors by human oligodendroglia. A. B. UNC5 (A, green) and DCC (B, green) immunoreactivity is detected in HF-OPCs, identified by expression of PDGFαR (A, red) and A2B5 (B, red). C, D, E. Netrin-1 (C, green) is detected in the cell bodies and process of HA-OPCs (A2B5, red). Unc5 homolog (D, green) and DCC (E, green) immunoreactivity associated with the extending processes and cell bodies HA-OPCs. F. DCC and UNC5 are detected along the processes of HA-OLGs. G. H. Netrin-1 (green) is distributed along the processes and in the sheets of MBP expressing HA-OLGs. (H) is a higher magnification of netrin-1 distribution (green) in the MBP-positive (red) membrane sheet (A, B, D, E, G: 40×0.75 n.a. objective. C, F: 20×0.5 n.a objective. H: 100×1.4 n.a. objective. Scale bars in A, B, G, H: 10 μm; C, O, E, F: 20 μm).

FIG. 14: Netrin-1 is present in Normal White Matter and MS lesions. A, i-v. DAB staining of sections of normal control white matter (NCWM1 and NCWM2). MS lesion and periplaque white matter (PPVVM)). i and ii. Arrows show netrin-1 immunoreactivity associated with oligodendrocytes (arrowheads) and astrocytes (arrows) in NCWM. iii and iv. Netrin-1 is detected in macrophages (asterisk), astrocytes (arrowheads) and oligodendrocytes (arrows) in MS lesions and PPWM. Diffuse netrin-1 immunoreactivity is present in the extracellular matrix (ECM). v. Higher magnification images of MS lesions showing netrin-1 associated with astrocytes and oligodendrocytes. vi. Immunofluorescence showing the netrin-1 protein associated with astrocytes and oligodendrocytes. B. SDS PAGE and western blot analysis of post-mortem tissue samples confirming the presence of netrin-1 in NCWM and MS lesions, and indicates the presence of full-length (arrows) and fragmented netrin-1 (arrowheads). C. Increase in abundance of a lower molecular weight netrin-1 immunoreactive band as development progresses in the rodent spinal cord (from Manitt, C., et al., 2001, J. Neurosci. 21:3911-3922).

FIG. 15: Full length and truncated netrin-1 have distinct functional effects on rodent and human oligodendroglia. A. Netrin-1 (100 ng/ml), but not netrin-1ΔC (100 ng/ml), induces an increase in the length of commissural axons extending from dorsal spinal cord explants. Addition of netrin-1 and netrin-1ΔC in combination results in reduced axon outgrowth. B. Addition of netrin-1ΔC (100 ng/ml) to the bottom chamber of a microchemotaxis assay decreases the number of rat Ops (ROPs) migrating through the transwell filter (Netrin-1ΔC bottom) to levels comparable with full length netrin-1 (Netrin-1 bottom). Addition of netrin-1ΔC to the top of the transwell filter and bottom of the microchemotaxis well (netrin-1ΔC top and bottom) repels ROPs. A combination of full length and shorter netrin-1 (Netrin-1 and Netrin-1ΔC bottom) does not decrease migration more than the either cue used alone. C. A dose response analysis of the effect of netrin-1ΔC on migrating ROPs shows maximal repulsion at 100 ng/ml. D. Netrin-1 (100 ng/ml) induces a decrease in the length of the longest HF-OPC process. The netrin-1 dependent decrease in process length is blocked by the addition of a DCC function blocking antibody (DCC-fb). E. Addition of netrin-1 and netrin-1ΔC (100 ng/ml) to HA-OPCs increased the length of OPC process. Blocking DCC function disrupted the process increasing effect of netrin-1, but not netrin-1ΔC. F. Addition of netrin-1 (100 ng/ml) induced a DCC-dependent increase in the surface area of HA-OLGs, but netrin-1ΔC did not have an effect on the cells. (*=p<0.05 vs. Control, *** p<0.001 vs. Control, y=p<0.05 vs. Netrin-1).

FIG. 16: Inhibition of U87 Glioblastoma Cell Migration by netrin-1 peptides. Peptides corresponding to three different highly conserved sequences found in domains VI and V of netrin-1 were synthesized (musc-1, musc-3 and musc-5). The peptides were added in media to the bottom well of Boyden chamber transfilter micochemotaxis assays, as illustrated in FIG. 3C. Cells (U87 human glioblastoma cell line) were plated in the top chamber and the number of cells migrating across the porous membrane, from the top side to the bottom side, were counted. The control condition reveals the spontaneous migration of the cells, moving in the absence of any added cue. Peptides were added to the bottom well at concentrations of (1) 1 ug/ml, (2) 500 ng/ml, and (3) 100 ng/ml; * indicates P<0.05 compared to control.

FIG. 17: Netrin-1 and the netrin-1 VI-V peptide inhibit angiogenesis in the retina during development. Panel (A) illustrates the normal vascularisation of the retina from post-natal day 1 (P1) to post-natal day 8 (P8). The photo-micrographs show retinal flatmounts stained with TRITC-labeled lectin to visualize the vasculature. Panel (B) illustrates the inhibition of retinal vessel growth by netrin-1 and the netrin-1 VI-V peptide. Netrin-1 and netrin-1 VI-V peptide were injected intravitreously at an estimated intraocular concentration of 100 ng/ml to 1 ug/ml on post-natal day 1 (P1). Retinal vascularisation was assessed at P4 and found to be substantially diminished. Images are representative of 3-4 independent experiments. The dotted line depicts the developing vascular front. Scale bar=1 mm. Data represent mean±SEM retinal vascularized areas, relative to that of vehicle-treated rats. * P<0.05.

FIG. 18. Netrin-1 and the netrin-1 VI-V peptide inhibit vascular sprouting ex vivo. In panel (A) micrographs illustrate sections of aortae dissected from adult C57BL6 mice, then cut into 1 mm thick rings and embedded in growth factor-reduced Matrigel (BD Biosciences) in 24-well tissue culture plates. Microvascular sprouting growing out of the rings was quantified by measuring the area covered by outgrowth of the aortic ring using ImagePro Plus 4.5 (Media Cybernetics, Silver Spring, Md.). Aortic rings were treated with netrin-1 or the netrin-1 VI-V peptide, either VI-V alone, or a VI-V-Fc protein chimera. Significantly reduced vascular growth was found in all three treatment groups. Scale bar=1 mm. Graphs represent mean±SEM, *P<0.05. All images are representative of 3-4 separate experiments. In panel (B) Netrin-1 and the netrin-1 VI-V peptide are shown to inhibit endothelial cell proliferation. Human retinal endothelial cells (HRECs) were obtained from Cell Systems (Kirkland, USA) and used from passage 2-7. Cell number was determined using the MIT (3-(4,5-dimethylthiazol-2-yl)-2,5-diphenyltetrazolium bromide) assay as described (Brault. S., et al., Am J Physiol Regul Integr Comp Physiol 292, R1174-1183 (2007)). Netrin-1 and the netrin VI-V peptide, either VI-V alone, or a VI-V-Fc protein chimera were introduced to media upon seeding and cell viability assessed after 36 hours.

### DETAILED DESCRIPTION

The invention described herein is based, at least in part, on the novel and unexpected finding that derivatives of netrin, and compounds derived therefrom, selectively inhibit cell growth, migration and/or branching.

### Compounds and Compositions of the Invention

Polypeptide sequences derived from netrin which demonstrate the selective inhibition of cell growth, migration and/or branching are disclosed herein. In one aspect, polypeptide sequences comprising the VI-V domain of netrin are provided herein. These polypeptides are also referred to as netrinΔC polypeptides (as the C domain of netrin is deleted). Fragments, derivatives, analogs, and modifications of these polypeptides which retain function as selective inhibitors of cell growth, migration and/or branching (also referred to as functional derivatives), are also encompassed.

In one embodiment, the subject polypeptide of the invention comprises the VI-V domain of a netrin, the VI domain of a netrin, a VI-Fc fusion, or a fragment, derivative, analog, or modification, or functional derivative thereof. Smaller peptides, for example peptides comprising a 15 to 30 amino acid region, or a 20 to 25 amino acid region. Fc fusions thereof, or derivatives, analogs, or modifications, or functional derivatives, thereof are also included.

Netrin sequences have been isolated from many species, including chick, fruit fly (e.g. Drosophila), nematode (e.g. C. elegans), mouse and human. Netrin sequences are highly conserved among species, including among vertebrates. It is contemplated that the netrin-derived sequence from any species may be used in the methods and compositions of the present invention. In an aspect, it is contemplated that the netrin-derived sequence from any vertebrate may be used in the methods and compositions of the present invention. In a preferred embodiment, the subject polypeptide of the invention is derived from the human netrin sequence.

Netrin proteins are highly conserved. For example, the chicken full length netrin-1 amino acid sequence is 89% identical to Human netrin-1, 89% identical to mouse netrin-1, and 50% identical to C. elegans UNC6. Chicken netrin-1 is 52% identical to Human netrin-3 and 52% identical to Mouse netrin-3. For Domains VI and V, the amino acid sequence identity is as follows: Chicken netrin-1 domain VI-V has 90% identity to Human netrin-1 domain VI-V; Chicken netrin-1 domain VI-V is 90% identical to Mouse netrin-1 domain VI-V; and Chicken netrin-1 domain VI-V is 58% identical to C. elegans Unc6 domain VI-V. For Domain VI, the amino acid sequence identity is as follows: Chicken netrin-1 domain VI has 88% identity to Human netrin-1 domain VI; Chicken netrin-1 domain VI is 87% identical to Mouse netrin-1 domain VI; and Chicken netrin-1 domain VI is 48% identical to C. elegans Unc6 domain VI.

The sequences of different netrins (such as netrin-1, netrin-2, netrin-3 and netrin-4) are also similar to each other. It is contemplated that the netrin-derived sequence or polypeptide of the invention may be derived from any netrin sequence, for example netrin-1, netrin-2, netrin-3 or netrin-4.

Representative netrin sequences and polypeptides of the invention are shown below:

Chicken netrin-1: (Accession no. NP_990750)
(SEQ ID NO: 1)
MPRRGAEGPLALLLAAAWLAQPLRGGYPGLNMFAVQTAQPDPCYDEHGLPRRCIPDFVNSAFGKEVKVSS
TCGKPPSRYCVVTEKGEEQVRSCHLCNASDPKRAHPPSFLTDLNNPHNLTCWQSDSYVQYPHNVTLTLSL
GKKFEVTYVSLQFCSPRPESMAIYKSMDYGKTWVPFQFYSTQCRKMYNKPSRAAITKQNEQEAICTDSHT
DVRPLSGGLIAFSTLDGRPTAHDFDNSPVLQDWVTATDIKVTFSRLHTFGDENEDDSELARDSYFYAVSD
LQVGGRCKCNGHASRCVRDRDDNLVCDCKHNTAGPECDRCKPFHYDRPWQRATAREANECVACNCNLHAR
RCRFNMELYKLSGRKSGGVCLNCRHNTAGRHCHYCKEGFYRDLSKPISHRKACKECDCHPVGAAGQTCNQ
TTGQCPCKDGVTGITCNRCAKGYQQSRSPIAPCIKIPAAPPPTAASSTEEPADCDSYCKASKGKLKINMK
KYCKKDYAVQIHILKAEKNADWWKFTVNIISVYKQGSNRLRRGDQTLWVHAKDIACKCPKVKPMKKYLLL
GSTEDSPDQGIIADKSSLVIQWRDTWARRLRKFQQREKKGKCRKA
Mouse netrin-1: (Accession no. 009118)
(SEQ ID NO: 2)
MMRAVWEALAALAAVACLVGAVRGPGLSMFAGQAAQPDPCSDENGHPRRCIPDFVNAAFGKDVRVSSTCG
RPPARYCVVSERGEERVRSCHLCNSSDPKKAHPPAFLTDLNNPHNLTCWQSENYLQFPHNVTLTLSLGKK
FEVTYVSLQFCSPRPESMAIYKSMDYGRINVPFQFYSTQCRKMYNRPHRAPITKONEQEAVCTDSHEDMR
PLSGGLIAFSTLEGRPSAHDFDNSPVIODWVTATDIRVAFERLETEIMENEDDSELARDSYYYAVSDLQV
GGRCKCNGHAARCVRDRDDSLVCDCRHNTAGPECDRCKPFHYDRPWQRATAREANECVACNCNLHARRCR
FNMELYKLSGRKSGGVCLNCRHNTAGRHCHTCKEGFYRDMGKPITHRKACKACDCHPVGAAGKTCNQTTG
QCPCKDGVTGITCNRCAKGYQQSRSPIAPCIKIPVAPPTTAASSVEEPEDCDSYCKASKGKLKMNMKKYC
RKDYAVQIHILKADKAGDWWKFTVNIISVYKQGTSRIRRGDQSLWIRSRDIACKCPKIKPLKKYLLLGNA
EDSPDQSGIVADKSSLVIQWRDTWARRLRKFQQREKKGKCKKA
Mouse netrin-3: (Accession. no. NP035077)
(SEQ ID NO: 3)
MPTWLWGLLLTAGTLSAALSPGLPASADPCYDEAREPRSCIPGLVNAALGREVLASSTCGRSANRVCDSSDPQRAHSA
DLLTSAPGTASPLCWRSDLLQQAPFNVTLTVPLGKAFELVFVSLRFCSAPPTSVALLKSQDHGRSWVPLGFFSSSCTL
DYGRLPAPADGPSGPGPEALCFPAPQAQPDGGGLLAFSVQDGSPQGLDLDNSPVLQDWVTATDIRIVLTRPAIQGDTR
DGGVTVPYSYSATELQVGGRCKCNGHASRCLLDTHGHLVCDCQHGTEGPDCSRCKPFYCDRPWQRATGQEAHACLACS
CNGHARRCRFNMELYRLSGRRSGGVCLNCRHNTAGRHCHYCREGFYRDPGRVLSDRRACRACDCHPVGAAGKTCNQTT
GQCPCKDGVTGLTCNRCAPGFQQSRSPVAPCVKTPVPGPTEESSPVEPQDCESHCRPARGSYRISLKKFCRKDYAVQV
AVGARGEARGSWTRFPVAVLAVFRSGEERARRGSSALWVPTLDAACGCPRLLPGRRYLLLGGGPGAAAGSTAGRGQGL
SAARGSLVLPWRDAWTRRLRRLQRRERRGRCGTA
Chicken netrin-2: (Accession no. AAA61743)
(SEQ ID NO: 4)
LRLLLTTSVLRLARAANPFVAQQTPPDPCYDESGAPRRCIPEFVNAAFGKEVQASSTCGKPPTRHCDASDPRRAHPPA
YLTDLNTAANMTCWRSETLHHLPHNVTLTLSLGKKFEVVYVSLQFCSPRPESTAIFKSMDYGKTWVPYQYYSSQCRKI
YGKPSKATVTKQNEQEALCTDGLTDLYPLTGGLIAFSTLDGRPSAQDFDSSPVLQDWVTATDIRVVFSRPHLFRELGG
REAGEEDGGAGATPYYYSVGELQVGGRCKCNGHASRCVKDKEQKLVCDCKHNTEGPECDRCKPFHYDRPWQRASAREA
NECLACNCNLHARRCRFNMELYKLSGRKSGGVCLNCRHNTAGRHCHYCKEGFYRDLSKSITDRKACKACDCHPVGAAG
KTCNQTTGQCPCKDGVTGLTCNRCAKGFQQSRSPVAPCIKIPAINPTSLVTSTEAPADCDSYCKPAKGNYKINMKKYC
KKDYVVQVNILEMETVANWAKFTINILSVYKCRDERVKRGDNFLWIHLKDLSCKPKIQISKKYLVMGISENSTRDRPG
LMADKNSLVIQWRDAWTRRLRKLQRREKKGKCVKP
Mouse netrin-4: (Accession no. NP_061295)
(SEQ ID NO: 5)
MGSCARLLLLWGCSAVAAGLNGVAGANSRCEKACNPRMGNLALGRKLRADTMCGQNATELFCFYSENADLTCRQPKCD
KCNAAHSHLAHPPSAMADSSFRFPRTWWQSAEDVHREKIQLDLEAEFYFTHLIMVFKSPRPAAMVLDRSQDFGKTWKP
YKYFATNCSATFGLEDDVVKKGAICTSRYSNPFPCTGGEVIFRALSPPYDIENPYSAKVQEQLKITNLRVRLLKRQSC
PCINDLNAKPHHFMHYAVYDFIVKGSCFCNGHADQCLPVEGFRPIKAPGAFHVVHGRCMCKHNTAGSHCQHCAPLYND
RPWEAADGRTGAPNECRTCKCNGHADTCHFDVNVWEASGNRSGGVCNNCQHNTEGQHCQRCKPGFYRDLRRPFSAPDA
CKACSCHPVGSAILPFSSVTFCDPSNGDCPCKPGVAGPHCDRCMVGYWGFGDYGCRPCDCAGSCDPLTGDCISSNADV
DWYHEVPTFHSMHNKSEPSWEWEDEQGFSALRHSGKCECKEQVLGNPKAFCGMKYSYVLKIKILSAHDKGSHAEVNVK
IKKVLKSTKLKILRGKRTLYPESWTNRGCTCPILNPGLEYLVAGHEDVRTGKLIVNMKSFVQHWKPALGRRVMHILKR
DCV
Human netrin-1: (Accession no. XM_044705)
(SEQ ID NO: 6)
MMRAVWEALAALAAVACLVGAVRGGPGLSMFAGQAAQPDPCSDENGHPRRCIPDFVNAAFGKDVRVSSTC
GRPPARYCVVSERGEERLRSCHLCNASDPKKAHPPAFLTDLNNPHNLTCWQSENYLQFPHNVTLTLSLGK
KFEVTYVSLQFCSPRPESMAIYKSMDYGRTWVPFQFYSTQCRKMYNRPHRAPITKQNEQEAVCTDSHTDM
RPLSGGLIAFSTLDGRPSAHDFDNSPVLQDWVTATDIRVAFSRLHTFGDENEDDSELARDSYFYAVSDLQ
VGGRCKCNGHAARCVRDRDDSLVCDCRHNTAGPECDRCKPFHYDRPWQRATAREANECVACNCNLHARRC
RFNMELYKLSGRKSGGVCLNCRHNTAGRHCHYCKEGYYRDMGKPITHRKACKACDCHPVGAAGKTCNQTT
GQCPCKDGVTGITCNRCAKGYQQSRSPIAPCIKIPVAPPTTAASSVEEPEDCDSYCKASKGKLKINMKKY
CKKDYAVQIHILKADAKAGDWWFTVNIISVYKQGTSRIRRGDQSLWIRSRDIACKCPKIKPLKKYLLLGN
AEDSPDQSGIVADKSSLVIQWRDTWARRLRKFQQREKKGKCKKA
Human netrin-3, also called human netrin-2 like (NTL2) (Accession no. 086758.1)
(SEQ ID NO: 7)
MPOWPWOLLLTAGTLFAALSPGPPAPADPCHDEGGAPRGCVPGLVNAALOREVLASSTCGRPATRACDAS
DPRRAHSPANNTSPOGTASPLCWRSESLPRAPLNVTLTVPLGKAFELVEVSLRFOSAPPASVALLKSQDH
GRSWAPLOFFSSHCDLDYGRLPAPANGPAGPGPEALCFPAPLAUDGSGTELAFSNQDSSPPOLDLDSSPV
SQDWVTATDVRVVLTRPSTAGDPRDMEAVVEYSYAATDLQVGGRCKCNGHASRCLLDTQGHLICDCRHGT
EGPDOORCKPFECDRPWQRATARESHACLACSCNGHARRCRFNMELYRLSGRRSGGVCLNCRHNTAGRHC
HYCREGFYRDPGRALSDRRACRACDCHPVGAAGKTCNQTTGQCPCKDGVTGLTCNRCAPGFQQSRSPVAP
CVKIPIPOPTEDSSPVQPQDCDSHOKPARGSYRISLKWECKKDYAVQVAVGARGEARGAWTREPVAVLAV
FRSGEERARRGSSALWVPAGDAACGCPRNLPGRRYLLLGGGPGAAAGGAGGRGPGLIAARGSLVLPWRDA
WTRRLRRIQRRERRGRCSAA
Human netrin-4: (Accession no. XM 031898)
(SEQ ID NO: 8)
MGSCARLLLLWGCTVVAAGLSGVAGVSSRCEKACNPRMGNLALGRKLWADTTCGQNATELYCFYSENTDL
TCRQPKCDKCNAAYPHLAHLPSAMADSSFRFPRTWWQSAEDVHREKIQLDLEAEFYFTHLIVMFKSPRPA
AMVLDRSQDFGKTWKPYKYFATNCSATFGLEDDVVKKGAICTSKYSSPFPCTGGEVIFKALSPPYDTENP
YSAKVQEQLKITNLRVQLLKRQSCPCQRNDLNEEPQHFTHYAIYDFIVKGSCFCNGHADQCIPVHGFRPV
KAPGTFHMVHGKCMCKHNTAGSHCQHCAPLYNDRPWEAADGKTGAPNECRACKCNGHADTCHFDVNVWEA
SGNRSGGVCDDCQHNTEGQYCQRCKPGFYRDLRRPFSAPDACKPCSCHPVGSAVLPANSVTFCDPSNGDC
PCKPGVAGRRCDRCMVGYWGFGDYGCRPCDCAGSCDPITGDCISSHTDIDWYHEVPDFRPVHNKSEPALG
VGGCAGVFCTSTLR
Domain VI and V
Chicken Processed domain VI-V (w/o signal peptide): 429 amino acids
(SEQ in NO: 9)
GYPGLNMFAVQTAQPDPCYDEHGLPRRCIPDFVNSAFGKEVKVSSTCGKPPSRYCVVTEKGEEQVRSCHL
CNASDPKRAHPPSFLTDLNNPHNLTCWQSDSYVQYPHNVTLTLSLGKKFEVTYVSLQFCSPRPESMAIYK
SMDYGKTWVPFQFYSTQCRKMYNKPSRAAITKQNEQEAICTDSHTDVRPLSGGLIAFSTLDGRPTAHDFD
NSPVLQDWVTATDIKVTFSRLHTFGDENEDDSELARDSYFYVASDLQVGGRCKCNGHASRCVRDRDDNLV
CDCKHNTAGPECDRCKPFHYDRPWQRATAREANECVACNCNLHARRCRFNMELYKLSGRKSGGVCLNCRH
NTAGRHCHYCKEGFYRDLSKPISHRKACKECDCHPVGAAGQTCNQTTGQCPCKDGVTGITCNRCAKGYQQ
Human Processed domain VI-V (w/o signal peptide): 429 amino acids
(From Accession no. NP_004813)
(SEQ ID NO: 10)
GGPGLSMFAGQAAQPDPCSDENGHPRRCIPDFVNAAFGKDVRVSSTCGRPPARYCVVSERGEERLRSCHL
CNASDPKKAHPPAFLTDLNNPHNLTCWQSENYLQFPHNVTLTLSLGKKFEVTYVSLQFCSPRPESMAIYK
SMDYGKTWVPFQFYSTQCRKMYNKPSRAAITKQNEQEAVCTDSHTDVRPLSGGLIAFSTLDGRPTAHDFD
NSPVLQDWVTATDIRVAFSRLHTFGDENEDDSELARDSYFYVASDLQVGGRCKCNGHASRCVRDRDDNLV
CDCRHNTAGPECDRCKPFHYDRPWQRATAREANECVACNCNLHARRCRFNMELYKLSGRKSGGVCLNCRH
NTAGRHCHYCKEGYYRDLSKPISHRKACKECDCHPVGAAGQTCNQTTGQCPCKDGVTGITCNRCAKGYQQ
SRSPIAPCI
Domain VI alone
Chicken Domain VI (w/o signal peptide): 260 amino acids
(SEO ID NO: 11)
GYPGENMFAVQTAQPDPCYDEHGLPRRCIPDFVNSAFGKEVKVSSTOGEPPSRYCVVTENGEEQVRSCHL
ONASDPKRAHETSFLTDANNPHNLTOWQSDSYVQYPHNVTLTESEGNKFEVTYVSLQFCSPRPESMAIYK
SMDYGKTWVPFQFYSTQCRKMYNKPSRAAITKQNEQEAICTDSHTDVRPLSGGLIAFSTLDGRPTAHDFD
NSPVLPDTAVTATDINVITSRLHTFGDENEDDSELARDSYFYAVSDLQVGG
Human Processed domain VI (w/o signal peptide): 260 amino acids
(From Accession no. NP 004813)
(SEQ ID NO: 12)
GGPGLSMFAGQAAQPDPCSDENGHPRRCIPDFVNAAFGKDVRVSSTCGRPPARYCVVSERGEERLRSCHL
CNASDPKKAHPPAFLTDLNNPHNLTCWQSENYLQFPHNVTLTLSLGKKFEVTYVSLQFCSPRPESMAIYK
SMDYGRTWVPFQFYSTQCRKMYNRPHRAPITKQNEQEAVCTDSHTDMRPLSGGLIAFSTLDGRPSAHDFD
NSPVLQDWVTATDIRVAFSRLHTFGDENEDDSELARDSYFYAVSDLQVGG
Shorter peptides
Musc-1 peptide sequence:
(SEQ ID NO: 13)
KPITHYDRPWQRATAREANEC
Musc-3 peptide sequence:
(SEQ ID NO: 14)
IYKSMDYGRTWVPF
Musc-5 peptide sequence:
(SEQ ID NO: 15)
RENMELYKLSGRKSOGVC
Domain VI sequences
(SEQ ID NO: 16)
ITKQNEOEAV
(SEQ ID NO: 17)
TDSIITDMR
(SEQ ID NO: 18)
SAHDFNDNSPVLQDWVTATDIE
Domain V sequences
(SEQ ID NO: 19)
KEGYYRDMGKPITHRKAC
(SEQ ID NO: 20)
AKGYQQSRSPIAPC

Non-limiting examples of other netrin sequences which may be used to derive the polypeptides of the invention are as follows: Human netrin-1 (for example, sequences of database accession nos. NP_{—}004813 and AAD09221); Mouse [Mus musculus] netrin 1 (e.g., sequences of database accession nos. NP_{—}032770.2, XP_{—}001480037.1, CAI25691.1, CAI24775.1, CAI25793.1, EDL10446.1, AAI41295.1, Locus 009118, AAD28602 and AAC52971); and Chicken [Gallus gallus ] netrin-1 (e.g. sequences of database accession nos. NP_{—}990750.1, 090922.1, AAA60369.1). It is contemplated that the domain VI-V region, domain VI region, and fragments thereof, from any netrin sequence from any species would retain the ability to selectively inhibit cell growth and/or migration and lack the ability of full-length netrin to also promote cell migration and therefore be useful in the methods and compositions of the invention.

The musc-1, musc-3 and musc-5 peptides correspond to three different highly conserved sequences found in domains VI and V of netrin-1. The musc-1 sequence is derived from domain VI of netrin-1 and is 100% identical in mouse, human and rat netrin-1. The musc-3 sequence is derived from domain VI of netrin-1 and is 100% identical in mouse and human, while in chicken, 13/14 amino acids are identical (92% identical). The musc-5 sequence is derived from sequence present in domain V and is 100% identical in mouse, human and rat netrin-1.

It is contemplated that any fragment, derivative, analog, or modification of a netrin sequence or of the VI-V domain or of the VI domain derived from a netrin which retains function as a selective inhibitor of cell migration, including shorter peptides such as those described herein, may be used in the compositions and methods described herein and is encompassed by the present invention.

The polypeptides disclosed herein may be free or covalently coupled to other atoms or molecules. Frequently the peptides are present as a portion of a larger polypeptide comprising the subject peptide. The invention provides polypeptides comprising a sequence substantially similar to that of the sequences disclosed herein. “Substantially similar” sequences share at least about 40%, preferably at least about 50%, more preferably at least about 60%, and most preferably at least about 80% sequence identity. Where the sequences diverge, the differences are generally point insertions/deletions or conservative substitutions, for example a cysteine/threonine or serine substitution, or an acidic/acidic or hydrophobic/hydrophobic amino acid substitution.

The subject polypeptides of the invention may be “isolated”, meaning that they are unaccompanied by at least some of the material with which they are associated in their natural state. Generally, an isolated polypeptide constitutes at least about 1%, at least about 5%, at least about 10%, or at least about 50% by weight of the total polypeptide in a given sample. By “pure” peptide or polypeptide is intended at least about 60%, preferably at least 80%, and more preferably at least about 90% by weight of total polypeptide. Included in the subject polypeptide weight are any atoms, molecules, groups, etc. covalently coupled to the subject polypeptides, such as detectable labels, glycosylations, phosphorylations, polypeptides which are covalently linked, and so on.

The subject polypeptides may be isolated or purified in a variety of ways known to those skilled in the art depending on what other components are present in the sample and to what, if anything, the polypeptide is covalently linked. Purification methods include electrophoretic, molecular, immunological and chromatographic techniques, especially affinity chromatography and RP-HPLC in the case of peptides. For general guidance in suitable purification techniques, see Scopes, R., Protein Purification, Springer-Verlag, N.Y. (1982).

The subject polypeptides generally comprise naturally occurring amino acids but D-amino acids or amino acid mimetics coupled by peptide bonds or peptide bond mimetics may also be used. Amino acid mimetics are other than naturally occurring amino acids that conformationally mimic the amino acid. Suitable mimetics are known to those of ordinary skill in the art and include for example beta, gamma and delta amino and imino acids, cyclohexylalanine, adamantylacetic acid, modifications of the amide nitrogen, the a-carbon, amide carbonyl, and backbone modifications (See, generally, Morgan and Gainor (1989) Ann. Repts. Med. Chem. 24, 243-252). Derivatives or modifications may also have one or more residues chemically derivatized by reaction of a functional side group Also included as derivatives or modifications are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

The subject polypeptides have selective activity as inhibitors of cell growth, migration or branching, meaning that the subject polypeptide does not also promote migration or evoke a chemoattractant response (in contrast to full-length netrin proteins which demonstrate a dual function as both promoters and repellants of cell migration). It should be understood that modified polypeptides (e.g. substitutions, additions, deletion, and so on), fragments thereof, or analogs thereof which retain this selective inhibitory function are encompassed herein. In an embodiment, polypeptides having at least 70%, at least 75%, at least 80%, at least 85%, at least 90%, and/or at least 95% sequence identity to the subject polypeptides of the invention are encompassed. For example, polypeptides having at least 75%, at least 80%, at least 85%, at least 90%, and/or at least 95% sequence identity to a netrin sequence, the sequence of the netrin VI-V domain, the sequence of the netrin VI domain, the sequence of the netrin V domain, the sequence of musc-1, the sequence of musc-3, the sequence of musc-5, and/or a fragment thereof, are encompassed herein. In an embodiment, the polypeptide or fragment thereof retains function as a selective inhibitor of cell migration.

In addition, the subject polypeptides may be modified or joined to other compounds using physical, chemical, and molecular techniques known to those skilled in the art. It is understood that properties such as solubility, membrane transportability, stability, toxicity, bioavailability, localization, detectability, in vivo half-life, and so on, may be affected by such modifications. Methods and assays for measuring such properties are known to those of ordinary skill in the art. For example, point mutations may be introduced by site directed mutagenesis of nucleotides in the DNA encoding the disclosed polypeptides or in the course of in vitro peptide synthesis. Other modifications to further modulate peptides include chemical/enzymatic intervention (e.g. fatty acid-acylation, proteolysis, glycosylation) and especially where the polypeptide is integrated into a larger polypeptide, selection of a particular expression host. Amino and/or carboxyl termini may be functionalized e.g., for the amino group, acylation or alkylation, and for the carboxyl group, esterification or amidification, or the like. For therapeutic and diagnostic localization, the subject polypeptides thereof may be labeled directly (radioisotopes, fluorescent labels, etc.).

In one embodiment, the subject polypeptides of the invention may be covalently linked to an Fc domain. Antibodies comprise two functionally independent parts, a variable domain known as “Fab”, which binds antigen, and a constant domain known as “Fc”, which does not bind antigen and links to such effector functions as complement activation and attack by phagocytic cells. An Fc has a long serum half-life and it is known in the art that, when constructed together with a therapeutic protein, an Fc domain can provide a longer in vivo half-life to the therapeutic protein. An Fc may confer other advantageous properties on the therapeutic protein such as improved solubility. An Fc domain may be monomeric or multimeric. The original immunoglobulin source of the Fc domain is preferably human and may be any of the immunoglobulins, although IgG1 and IgG2 are generally preferred.

Accordingly, there are provided herein fusion proteins comprising the subject polypeptides of the invention covalently linked to an Fc domain. For example, the VI domain of netrin may be covalently linked to an Fc domain (termed “VI-Fc” or “domain VI-Fc”). Any of the peptide and polypeptide sequences encompassed herein may be covalently linked to an Fc domain.

Also provided herein are isolated nucleic acids encoding the subject polypeptides of the invention. An “isolated” nucleic acid is present as other than a naturally occurring chromosome or transcript in its natural state and is typically joined in sequence to at least one nucleotide with which it is not normally associated on a natural chromosome.

In an aspect, subject nucleic acids may be partially pure. A “partially pure” nucleotide sequence constitutes at least about 5%, or at least about 10%, or at least about 30%, or at least about 80%, or at least about 90% by weight of total nucleic acid present in a given fraction.

Nucleic acids with substantial sequence similarity are also encompassed. Such nucleic acids typically hybridize under low stringency conditions, for example, at 50° C. and SSC (0.9M saline/0.09M sodium citrate) and remain bound when subject to washing at 55° C. with SSC. In other embodiments, nucleic acids having at least 50% sequence identity, at least 60% sequence identity, at least 70% sequence identity, at least 80% sequence identity, at least 90% sequence identity or at least 95% sequence identity are encompassed herein. It will be understood by the person skilled in the art that due to the degeneracy of the genetic code, nucleic acids may be modified by deletions, insertions, or substitutions, for example, and still encode the subject polypeptides. In addition, nucleic acids of the invention may include additional non-coding sequences such as genomic sequences, or gene flanking sequences, including regulatory sequences such as promoters, enhancers, response elements, signal sequences, polyadenylation sequences, introns, 5′ and 3′ noncoding regions, and so on. According to a particular embodiment of the invention, portions of the coding sequence are spliced with heterologous sequences to produce soluble, secreted fusion proteins, using appropriate signal sequences and optionally, a fusion partner.

The netrin-derivative encoding nucleic acids can be subject to alternative purification, synthesis, modification, sequencing, expression, transfection, administration or other use by methods disclosed in standard manuals such as Molecular Cloning, A Laboratory Manual (2nd Ed., Sambrook, Fritsch and Maniatis, Cold Spring Harbor), Current Protocols in Molecular Biology (Eds. Aufubel, Brent, Kingston, More, Feidman, Smith and Stuhl, Greene Publ. Assoc., Wiley-Interscience, New York, N.Y., 1992) or that are otherwise known in the art.

Also provided herein are vectors comprising nucleic acids encoding the subject polypeptides of the invention. A large number of vectors, including plasmid and viral vectors, have been described for expression in a variety of eukaryotic and prokaryotic hosts. Advantageously, vectors will often include a promotor operably linked to the polypeptide-encoding portion, one or more replication systems for cloning or expression, or one or more markers for selection in the host, e.g. antibiotic resistance. The inserted coding sequences may be synthesized, isolated from natural sources, prepared as hybrids, and so on. Suitable host cells may be transformed/transfected/infected by any suitable method including electroporation, CaCl₂ mediated DNA uptake, viral infection, microinjection, microprojectile, or other methods known in the art.

The compounds of the invention may be used for therapeutic or prophylactic purposes by formulating them with appropriate pharmaceutical carrier materials and administering an effective amount to a patient, such as a human (or other mammal) in need thereof.

For therapeutic or prophylatic uses, the compounds, agents and compositions disclosed herein may be administered by any convenient way, as known in the art. Small organics are preferably administered orally; other compositions and agents are preferably administered parenterally, conveniently in a pharmaceutically or physiologically acceptable carrier, e.g., phosphate buffered saline, or the like. Many pharmaceutically acceptable carriers are known in the art and chosen according to route of administration and other particularities of the agents involved. Compositions may be added to a retained physiological fluid such as blood or synovial fluid. For CNS administration, a variety of techniques are available for promoting transfer of the therapeutic across the blood brain barrier including disruption by surgery or injection, drugs which transiently open adhesion contact between CNS vasculature endothelial cells, and compounds which facilitate translocation through such cells. In some cases, the therapeutic agents and compositions disclosed herein will be administered by injection or directly to the affected site, for example during surgery.

As examples, many of the disclosed therapeutics are amenable to direct injection or infusion, topical, intratracheal/nasal administration e.g. through aerosol, intraocularly, or within/on implants e.g. fibers e.g., collagen, osmotic pumps, grafts comprising appropriately transformed cells, etc. One application involves coating, imbedding or derivatizing fibers, such as collagen fibers, protein polymers, etc. with therapeutic peptides. Other useful approaches are described in Otto et at. (1989) J Neuroscience Research 22, 83-91 and Otto and Unsicker (1990) J Neuroscience 10, 1912-1921.

Generally, the amount administered will be empirically determined, typically in the range of about 10 to 1000 μg/kg of the recipient, For peptide agents, the concentration will generally be in the range of about 50 to 500 μg/ml in the dose administered. Other additives may be included, such as stabilizers, bactericides, etc. These additives will be present in conventional amounts. Pharmaceutically acceptable carriers may be added to a therapeutic according to standard procedures in the art.

The dosage or amount of the compounds, agents and compositions, e.g. subject polypeptides and polynucleotides, of the invention depends on the individual case and is, as is customary, to be adapted to the individual circumstances to achieve an optimum effect. Thus, it depends on the nature and the severity of the disorder to be treated, and also on the sex, age, weight and individual responsiveness of the human or animal to be treated, on the efficacy and duration of action of the compounds used, on whether the therapy is acute or chronic or prophylactic, or on whether other active compounds are administered in addition to the therapeutic compound, agent or composition. It shall be understood that dosing and administration regimens are within the purview of the skilled artisan.

A composition within the scope of the present invention should contain the active agent (e.g. polypeptide, fusion protein, or nucleic acid) in an amount effective to achieve the desired therapeutic effect. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted in accordance with conventional factors such as the extent of the disease and different parameters from the patient. Typically, 0.001 to 50 mg/kg/day will be administered to the mammal.

The pharmaceutical composition according to the invention can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or wafers.

The preparation of the pharmaceutical compositions can be carried out as known in the art For example, the subject polypeptides and polynucleotides, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human medicine. The pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

### DEFINITIONS AND TERMS

The terminology used herein is for the purpose of describing particular embodiments only and is not intended to limit the scope of the present invention.

Use of the singular forms “a,” “an,” and “the” include plural references unless the context clearly dictates otherwise. Thus, for example, reference to “a target polynucleotide” includes a plurality of target polynucleotides.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value. Where a value is explicitly recited, it is to be understood that values which are about the same quantity or amount as the recited value are also within the scope of the invention, as are ranges based thereon.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The terms “polynucleotide,” “oligonucleotide,” “nucleotide”, “nucleic acid” and “nucleic acid molecule” are used interchangeably herein to refer to a polymeric form of nucleotides of any length, and may comprise ribonucleotides, deoxyribonucleotides, analogs thereof, or mixtures thereof. These terms refer only to the primary structure of the molecule. Thus, the terms include triple-, double- and single-stranded deoxyribonucleic acid (“DNA”), as well as triple-, double- and single-stranded ribonucleic acid (“RNA”). They also include modified (for example, by alkylation and/or by capping) and unmodified forms of the polynucleotide. The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

As used herein, the designation “functional derivative” denotes, in the context of a functional derivative of a sequence whether a nucleic acid or amino acid sequence, a molecule that retains a biological activity (either function or structural) that is substantially similar to that of the original sequence, e.g. the ability to selectively inhibit cell growth, migration or branching. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophilicity and the like. The term “functional derivatives” is intended to include “fragments”, “segments”, “variants”, “analogs”, “modifications” or “chemical derivatives” of the subject matter of the present invention. The functional derivatives of the present invention can be synthesized chemically or produced through recombinant DNA technology. All these methods are well known in the art.

The term “selectively inhibit” or “selective inhibition” is used herein to refer to compounds, e.g. subject polypeptides and polynucleotides of the invention, which inhibit cell growth, migration or branching without evoking a chemoattractant response or promoting migration, i.e. which do not have the bifunctional activity of netrins but instead are specific for inhibiting cell growth, migration or branching.

As used herein, the terms “molecule”, “compound”, or “agent” are used interchangeably and broadly to refer to natural, synthetic or semi-synthetic molecules or compounds. The term “molecule” therefore denotes for example chemicals, macromolecules, cell or tissue extracts (from plants or animals) and the like. Non limiting examples of molecules include nucleic acid molecules, peptides, antibodies, carbohydrates, low molecular weight organic compounds, and pharmaceutical agents. The agents can be selected and screened by a variety of means including random screening, rational selection and by rational design using for example protein modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Similarly, in an embodiment, the polypeptides of the present invention are modified to enhance their stability.

### Therapeutic Uses

Netrins and netrin receptors are known to play a role in tumorigenesis. Netrin-1 is widely expressed by neurons and glia in the adult CNS (Manitt, C., et al., 2001, J. Neurosci. 21:3911-3922) and reduced expression has been documented in brain tumors, including glioblastoma (Meyerhardt. J. A., et al., 1999, Cell Growth Differ. 10:35-42). Substantial evidence points to an anti-oncogenic role for the netrin receptor DCC. Indeed, the dcc gene was first identified as a putative tumor suppressor from a chromosomal deletion (Fearon, E. R., et al., 1990. Science 247:49-56). Dcc expression is reduced in many cancers, including most high-grade gliomas (Ekstrand. B. C., et al., 1995. Oncogene 11:2393-2402; Reyes-Mugica, M., et al., 1997, Cancer Res. 57:382-386) and loss of DCC correlates with the development of highly invasive glioblastoma multiformae (Reyes-Mugica, M., et al., 1997, Cancer Res. 57:382-386). Reduced expression of UNC5A, B and C has also been detected in various cancers, suggesting that they may also function as tumor suppressors (Thiebault, K., et al., 2003. Proceedings of the National Academy of Sciences of the United States of America 100:4173-4178).

However, the bifunctional activity of netrins, attracting some cell types and repelling others, makes them unattractive candidates for treating or preventing disease associated with unwanted cell growth, migration or branching. In contrast, the selective inhibitory or repellant functions of the compounds disclosed herein indicates a role for these compounds in therapeutic and prophylactic treatment of diseases involving uncontrolled, excessive or inappropriate cell growth; migration and/or branching. The compounds disclosed herein may be used as inhibitors of tumorigenesis and/or angiogenesis, in particular neovascularization (NV), and may thus have broad therapeutic or prophylactic application. For example, deregulation of mechanisms that control cell motility plays a key role in tumor progression by promoting tumor cell dissemination. Existing therapies for glioblastoma poorly control cells migrating away from the tumor, leading to recurrence of the tumor following surgical resection. Ocular neovascularization is associated with the vast majority of eye diseases that lead to catastrophic loss of vision.

Angiogenesis is the process by which new blood vessels form. In response to specific chemical signals, capillaries sprout from existing vessels, eventually growing in size as needed by the organism. Angiogenesis is stimulated by a number of conditions, such as in response to a wound, and accompanies virtually all tissue growth in vertebrate organisms such as mammals. NV is often an abnormal or excessive proliferation and growth of blood vessels. The development of NV itself often has adverse consequences or it can be an early pathological step in disease. For example, NV is implicated in retinal neovascular diseases and in tumor progression.

ARMD and diabetic retinopathy are the leading causes of visual loss in industrialized nations and do so as a result of abnormal retinal neovascularization. Since the retina consists of well-defined layers of neuronal, glial, and vascular elements, relatively small disturbances such as those seen in vascular proliferation or edema can lead to significant loss of visual function. Inherited retinal degenerations, such as retinitis pigmentosa (RP), are also associated with vascular abnormalities, such as arteriolar narrowing and vascular atrophy.

Age related macular degeneration (ARMD) affects 12-15 million Americans over the age of 65 and causes visual loss in 10-15% of them as a direct effect of choroidal (sub-retinal) neovascularization. The leading cause of visual loss for Americans under the age of 65 is diabetes; 16 million individuals in the United States are diabetic and 40,000 per year suffer from ocular complications of the disease, often a result of retinal neovascularization. Inherited degenerations of the retina affect as many as 1 in 3500 individuals and are characterized by progressive night blindness, visual field loss, optic nerve atrophy, arteriolar attenuation, altered vascular permeability and central loss of vision often progressing to complete blindness (Heckenlively, J. R., editor, 1988; Retinitis Pigmentosa, Philadelphia: JB Lippincott Co.). Other eye diseases in which proliferation of new blood vessels plays a critical role include proliferative diabetic retinopathy, rubeotic glaucoma, interstitial keratitis and retinopathy of prematurity.

No treatment is currently available to specifically treat ocular vascular disease. There is a need for treatments to prevent visual loss, for example in patients with choroidal neovascularization due to ARMD or inflammatory eye disease such as ocular histoplasmosis. There are still no effective treatments to slow or reverse the progression of these retinal degenerative diseases. Moreover, treatment options for controlling NV are inadequate and a large and growing unmet clinical need remains for effective treatments of NV, either to inhibit disease progression or to reverse unwanted angiogenesis.

Angiogenesis also plays a central role in cancer. Sustained growth and metastasis of a variety of tumors has been shown to be dependent on the growth of new host blood vessels into the tumor. Tumors require blood vessel growth to provide oxygen and nutrients to the growing tumor tissue. In addition, NV in cancer correlates with cancer growth and metastasis. Therefore, the effective inhibition of neovascularization is thought to be one of the promising strategies for cancer therapy.

Accordingly, the compositions and methods provided herein can be used for the treatment of diseases such as cancer, ocular disease, and inflammatory diseases of the eye, which involve NV, the branching or growth of vasculature, or unwanted cell growth or migration.

Non-limiting examples of ocular disease which may be treated according to the compositions and methods provided herein include wet and dry age-related macular degeneration; retinal disorders, including without limitation, diabetic retinopathy, retinitis pigmentosa (RP), inflammatory disease including macular edema, central vein occlusion, uveitis affecting the retina, and proliferative vitreoretinopathy; rubeotic glaucoma; interstitial keratitis; retinopathy of prematurity; corneal neovascularization, i.e. inflammatory, transplantation, or developmental hypoplasia of the iris; neovascularization resulting following a combined vitrectomy and lensectomy; neovascularization of the optic nerve; and neovascularization due to penetration of the eye or contusive ocular injury.

Cancer refers herein to a cluster of cancer or tumor cells showing over-proliferation by non-coordination of the growth and proliferation of cells due to the loss of the differentiation ability of cells. The terms “cancer cell” and “tumor cell” are used interchangeably herein. The term “cancer” includes but is not limited to, colon cancer (generally considered the same entity as colorectal and large intestinal cancer), glioma, astrocytoma, glioblastoma multiforme, breast cancer, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, fallopian tube carcinoma, endometrial carcinoma, cervical cancer, vulval cancer, squamous cell carcinoma, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, primary CNS lymphoma, bone marrow tumor, brain stem nerve gliomas, pituitary adenoma, uveal melanoma (also known as intraocular melanoma), testicular cancer, oral cancer, pharyngeal cancer or a combination thereof. The term “cancer” also includes pediatric cancers, including pediatric neoplasms, including leukemia, neuroblastoma, retinoblastoma, glioma, rhabdomyoblastoma, sarcoma and other malignancies.

In an embodiment, the cancer is a brain tumor, e.g. glioma. In another embodiment, the cancer is colorectal cancer. In a particular embodiment, the invention relates to treatment of colorectal cancer, brain cancer, such as malignant glioma, such as Glioblastoma multiforme (GBM), breast cancer, or pancreatic cancer.

In one aspect, there is provided herein a method for treating or preventing cancer comprising administering an effective amount of the compounds and compositions of the invention to a subject in need thereof. The cancer may be, for example, colorectal cancer, brain cancer, malignant glioma. GBM, breast cancer or pancreatic cancer. In another aspect, there is provided herein a method for treating or preventing age-related macular degeneration, a retinal disorder, diabetic retinopathy, retinitis pigmentosa (RP), or an inflammatory disease of the eye.

In yet another aspect, there is provided a method for inhibiting angiogenesis or neovascularization in a subject in need thereof, comprising administering an effective amount of the compounds and compositions of the invention to the subject. In a further aspect, there is provided a method for inhibiting tumor growth or metastasis in a subject in need thereof, comprising administering an effective amount of the compounds and compositions of the invention to the subject.

In an aspect, the methods described herein comprise contacting a tumor cell undergoing or likely to undergo movement with a subject polypeptide of the invention. In another aspect, the methods described herein comprise contacting a tumor cell undergoing or likely to undergo movement with a subject polypeptide of the invention and a laminin polypeptide in an amount effective to inhibit, decrease or modulate migration of the tumor cell, thereby inhibiting, decreasing or modulating cell migration in the subject. In yet another aspect, there is provided herein a method of promoting the maturation of focal complexes (FCs) into focal adhesions (FAs) to inhibit or restrain tumor cell migration in a subject, the method comprising contacting a tumor cell undergoing or likely to undergo movement with a subject polypeptide of the invention, or with a subject polypeptide of the invention and a laminin polypeptide, in an amount effective to promote the maturation of FCs into FAs in the subject, thereby inhibiting or restraining cell migration.

In another aspect, there is provided herein a kit for performing any of the methods described herein. A kit typically comprises a subject polypeptide of the invention, or a polynucleotide encoding a subject polypeptide, or a composition containing a subject polypeptide or a polynucleotide, and instructions for use thereof.

The autoimmune disorder Multiple Sclerosis (MS) is characterized by the presence of focal regions of demyelination in the CNS. A model for the failure of remyelination proposes that the environment of a chronic lesion is not conducive for the recruitment of adult oligodendrocyte progenitors (OPCs) and the differentiation of these cells into mature, myelinating oligodendrocytes (OLGs) (Franklin R J, et al., 2008, Nat Rev Neurosci 9:839-855). Inhibitors of oligodendrocyte differentiation have been detected in myelin debris (Kotter M R, et al., 2006, J Neurosci 26:328-332) and the extracellular matrix (ECM) (Back et al., 2005, Nat Med 11:966-972), and may actively prevent myelination. Additionally, adult OPCs and OLGs in a demyelinated lesion may not be subject to the precise sequence of events that lead to the initiation of myelination during development, resulting in their failure to myelinate demyelinated axons. Netrin-1 is known to play multiple roles during oligodendrocyte development. In mature, myelinating oligodendrocytes, autocrine expression of netrin-1 is required for myelin-like membrane formation and the maintenance of axo-oligodendroglial paranodal junctions (Manitt. C., et al., 2001. Neurosci. 21:3911-3922; Jarjour A A, et al., 2008, J Neurosci 28:11003-11014).

We report herein that netrin-1 is present in MS lesions, associated with oligodendrocytes, astrocytes, macrophages and the ECM. We also detect an abundance of shorter fragments of netrin-1 lacking the C domain in lesions (the VI-V domain). Netrin-1 induces morphological changes in human OPCs and OLGs in vitro, but loss of the C domain results in an inability of netrin-1 to induce an increase in the surface area of human adult OLGs. Our data shows that a proteolytically cleaved fragment of netrin-1 present in MS lesions retains the ability to influence the migration of OPCs to the lesion site, but that these fragments cannot promote the final stages of morphological maturation leading to myelination.

Accordingly, in another aspect, there is provided a method for diagnosis or prognosis of multiple sclerosis (MS) in a subject in need thereof, comprising determining whether the VI-V fragment of netrin is present in the subject. For example, the VI-V fragment of netrin, or a derivative or fragment thereof, may be detected in the blood, in the CSF, or in a lesion in a subject.

### EXAMPLES

The present invention will be more readily understood by referring to the following examples which are given to illustrate the invention rather than to limit its scope.

### Example 1

### Glioblastoma Cells Express Netrin and Netrin Receptors

To determine if netrins regulate glioblastoma cell migration, we first characterized netrin and netrin receptor expression in human astrocytoma cell lines U87. U343, and U373, and in cultures of astrocytes isolated from newborn rat cortex (FIG. 3A). Western blot analysis using an antibody that binds netrin-1 and netrin-3 (Manitt. C., et al., 2001, J. Neurosci. 21:3911-3922) detected a ˜75 kDa band corresponding to full length netrin in conditioned medium collected from all cells tested. The DCCIN antibody detected a ˜185 kDa band, corresponding to DCC in astrocyte and U87 cell lysates. In contrast, DCC was not detected in lysates of U343 or U373 cells. The DCC homologue neogenin was expressed by astrocytes and all glioblastoma cell lysates. RT-PCR (FIG. 3B) revealed dcc expression by U87 cells but not U343 or U373 cells, and neogenin and unc5 homologue expression by all three cell types. U87 cells express only unc5b. U343 cells express unc5a, b, and c, and U373 cells express unc5a, b, C, and d. Netrin-1 expression was detected in U343 and U373 cells, and netrin-3 expression in U87 cells. Netrin-1 and netrin 3 both bind DCC and UNC5 homologues and similarly evoke chemoattractant or chemorepellent responses (Wang. H., et al., 1999, J. Neurosci. 19:4938-4947).

As a result of finding that these cells express both netrins and netrin receptors, we then sought to determine if netrin might exert an autocrine influence on cell migration. We first assessed the relative motility of the three cell lines using a transfilter chemotaxis assay as described (Jarjour, A. A., et al., 2003, J. Neurosci. 23:3735-3744). Briefly, cells were cultured on the upper surface of a porous membrane (FIG. 3C) and allowed to migrate across. Following migration, cells remaining on the upper surface of the membrane were scraped off, and the cells that migrated to the underside fixed, stained, and counted. While this assay is most commonly employed to assess the migration of cells in response to a putative attractant or repellent cue, here we used it in the absence of added factors to compare the relative rates of spontaneous migration of the three glioblastoma lines. U343 and U373 cells, lacking DCC, migrated significantly faster than DCC expressing U87 cells. Notably, the U343 cells, which were derived from a grade IV glioblastoma multiformae (Nister. M., et al., 1987, Cancer Res. 47:4953-4960), migrated significantly faster than either the U87 or U373 cells, both of which were derived from less aggressive grade III astrocytomas (Ponten. J. and E. H. Macintyre, 1968, Acta Pathol. Microbiol. Scand. 74:465-486).

### Example 2

### Netrins Inhibit Human Glioblastoma Cell Migration In Vitro

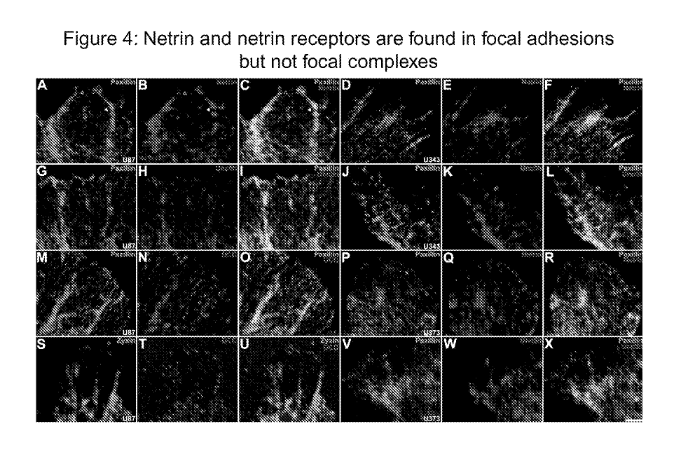

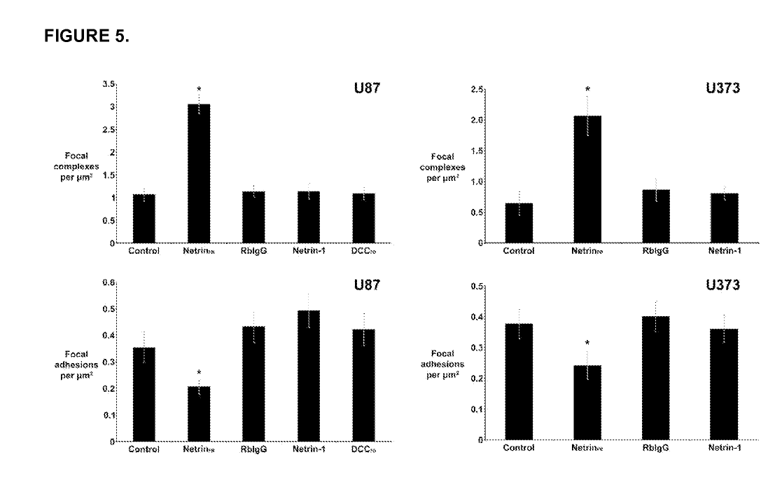

We have obtained evidence that netrins can function as autocrine factors that inhibit cell migration. Human glioblastoma cell lines (U87, U373) express netrin receptors and either netrin-1 or netrin-3 (FIG. 3). We routinely use transfilter microchemotaxis assays (FIG. 3C) to assess the rate of cell migration as described (Jarjour. A. A., et al., 2003, J. Neurosci. 23:3735-3744). Cells are plated on the upper side of a polycarbonate transwell culture inserts (6.5 mm diameter with 8 μm pore size. Corning) and allowed to migrate. Cells migrating into a pore are challenged with either an increasing gradient of a cue placed in the bottom compartment only, decreasing gradients of a cue place in the top only, or a uniform concentration of a cue placed in both the bottom and the top. The spontaneous rate of cell migration can be measured by assessing the number of cells that migrate in the absence of an added cue. Following migration, cells on the upper side of the filter are scraped off, and the cells attached to the lower side of the filter fixed, cell nuclei stained with Hoechst dye, and counted using automated software. Using transfilter migration assays as described (Jarjour, A. A., et al., 2003, J. Neurosci. 23:3735-3744), we have found that disrupting autocrine netrin function using a netrin function blocking antibody (FIG. 3, Net_{FB}) applied uniformly to the top and bottom wells causes a substantial increase in the rate of cell migration compared to controls with medium alone (FIG. 3D,E: U87 ˜25 fold increase, U373 ˜3 fold increase). Furthermore, netrin-1, netrin-3. DCC, and UNC5 homologues were detected in mature focal adhesions (FA), but not focal complexes (FC), transient adhesive structures associated with cell motility (FIG. 4). Consistent with increasing the rate of cell migration in the transfilter assay, disrupting endogenous netrin function promoted the formation of FCs (FIG. 5). These findings are consistent with a role for netrins regulating cell-cell and cell-ECM adhesion.

### Example 3

### Autocrine Netrin-1 Inhibits U87 and U373 Cell Motility

U87 cells, which express DCC, migrate substantially more slowly than either U343 or U373 cells, which do not express DCC (FIG. 3C). We hypothesized that netrin and DCC expressed by U87 cells might exert a kinetic influence on the rate of cell movement, independent of netrin's influence on directional migration. We therefore tested the effect of blocking DCC and netrin function on the spontaneous rate of U87 cell migration. Addition of netrin function-blocking antibody (Net_{FB}) to both the top and bottom compartments, thereby disrupting autocrine netrin function, resulted in a greater than 25 fold increase in spontaneous migration across the filter relative to the number of cells migrating in either medium alone (Control), or in the presence of a control IgG (FIG. 3D). In contrast, the rate of spontaneous migration was not affected by addition of DCC function-blocking antibody (DCC_{FB}).

### Example 4

### Autocrine Netrin-1 Inhibits Migration of U373, but not U343, Glioblastoma Cells

Netrin's capacity to inhibit U87 cell motility in a DCC-independent manner led us to determine if a similar mechanism was active in U343 or U373 cells, which do not express DCC. The addition of netrin function-blocking antibody to both the top and bottom compartments of the transfilter assay significantly increased the rate of U373 migration (FIG. 3E), indicating that endogenous netrin-1 inhibits the rate of U373 migration.

Unlike U87 and U373 cells, blocking netrin function did not alter the rate of U343 cell migration. Grade IV glioblastoma multiformae-derived U343 cells were the most rapidly migrating of the three cell types examined, and while they express neogenin and UNC5 homologue netrin receptors, the absence of an increase in the rate of migration may be the result of more severe disruption of the mechanisms that regulate the motility of these cells.

### Example 5

### Netrin-1 is a Chemotropic Attractant for U87 Glioblastoma Cells

Transfilter migration assays were then used to determine if DCC-expressing U87 cells respond to a gradient of netrin-1 as a chemoattractant. Addition of 100 ng/ml netrin-1 to the bottom compartment (NB) produced a significant increase in the number of U87 cells that migrated across the membrane relative to control (medium alone; FIG. 8A, 16 hr assay; FIG. 8B, 48 hr assay). In contrast, when netrin-1 was added to both the top and bottom compartments (NTB), migration was not significantly different from control. This indicates that U87 cells respond to a gradient of netrin-1 as a chemotropic attractant. When challenged with a gradient of netrin-1, with DCC_{FB} antibody in the top and bottom wells (NB DCC_{FB}), U87 cells migrated not significantly different from control, indicating that the tropic response of U87 cells to netrin-1 requires DCC. Neither U343 nor U373 cells, which do not express DCC, altered their migration in response to an exogenous gradient of netrin-1 (FIG. 8C), despite expressing neogenin and UNC5 homologue netrin receptors. These findings suggest that although these receptors may be sufficient to mediate autocrine inhibition of migration (FIG. 3E), they are insufficient for these cells to generate a chemotropic response to a gradient of netrin-1 (FIG. 8C).

### Example 6

### Chemoattractant Response of DCC-Expressing U343 and U373 Cells to a Gradient of Netrin-1

To further investigate the contribution of DCC to the regulation of cell motility, we reintroduced the dcc gene back into U343 and U373 cells by transfection with a cDNA encoding a DCC-GFP chimera (pDCC-GFP, described by Shekarabi. M. and T. E. Kennedy, 2002, Mol. Cell Neurosci. 19:1-17). Expression of DCC by U343 and U373 cells was confirmed by western Not (FIG. 3A). Unlike the parental U343 and U373 cells lines. DCC-GFP expressing U343D and U373D cells migrated up a gradient of netrin-1 (FIG. 8D, E), indicating that DCC transgene expression in these cells was sufficient to generate a chemotropic response to netrin-1. Like DCC-expressing U87 cells, the gain of function migration towards netrin-1 exhibited characteristics of true chemotropic attraction, as the cells only responded to a gradient. Uniform presentation of exogenous netrin-1 produced migration that was not significantly different from control. The DCCFB antibody blocked the chemoattractant response of U343D and U373D cells, indicating that DCC is required for chemoattraction to netrin-1.

Consistent with the slow migration of DCC expressing U87 cells, the number of DCC-transfected U343 and U373 cells that migrated under control conditions was substantially reduced relative to that of the parental cells (FIG. 8D, E). These findings suggest that DCC expression decreases the motility of these cells; however, again consistent with the U87 cells (FIG. 3D), application of the DCC function-blocking antibody (DCCFB) did not increase the rate of migration. In contrast, DCCFB completely blocked the chemoattractant migratory response of the U87 cells, and the DCC-transfected U343 and U373 cells to a gradient of netrin-1. These findings are consistent with DCC expression engaging a mechanism that slows non-directional cell migration, but that is insensitive to DCCFB.

### Example 7

### Chemoattraction to Netrin-1 is Converted to Repulsion by Laminin-1

Laminin-1 converts the response of Xenopus retinal ganglion cell growth cones to netrin-1 from attraction to repulsion (Hopker. V. H., et al., 1999. Nature 401:69-73). We therefore investigated the possibility that laminin-1 might influence the migratory response of U87 cells to a gradient of netrin-1 (FIG. 9). When U87 cells were challenged with an ascending gradient of laminin-1 (LB), the number of cells that migrated to the underside of the membrane increased. In a uniform concentration of infor (LTB). U87 migration was not significantly different from control, indicating that a gradient of laminin-1, like netrin-1, is a chemoattractant for these cells. Interestingly, the combination of an ascending gradient of netrin-1 and a uniform concentration of laminin-1 (LTBNB) dramatically reduced the number of U87 cells that migrated to the underside of the membrane, suggesting that laminin-1 converted netrin-1 from an attractant to a repellent. Consistent with this, confronting cells with a descending netrin-1 gradient in the presence of a uniform concentration of laminin-1 (LTBNT), resulted in an increase in migration relative to controls. When the cells were simultaneously exposed to uniform concentrations of netrin-1 and laminin-1, (LTBNTB), fewer cells migrated to the underside of the membrane, indicating that the combined action of netrin-1 and laminin-1 exert a non-directional effect that inhibits U87 cell motility. These results are consistent with laminin-1 switching netrin-1 from an attractant to a repellent for U87 cells, as previously described for the axons of Xenopus retinal ganglion cells (Hooker. V. H., et al., 1999. Nature 401:69-73). Addition of DCCFB antibody in the presence of a uniform concentration of laminin-1 and either an increasing gradient (LTBNB DCCFB) or uniform concentration (LTBNTB DCCFB) of netrin-1, produced migration that was not significantly different from control, indicating that the laminin-induced repellent response to netrin-1 requires DCC.

### Example 8

### Netrin-1 and Dcc do not Affect U87, U343, and U373 Cell Survival or Proliferation

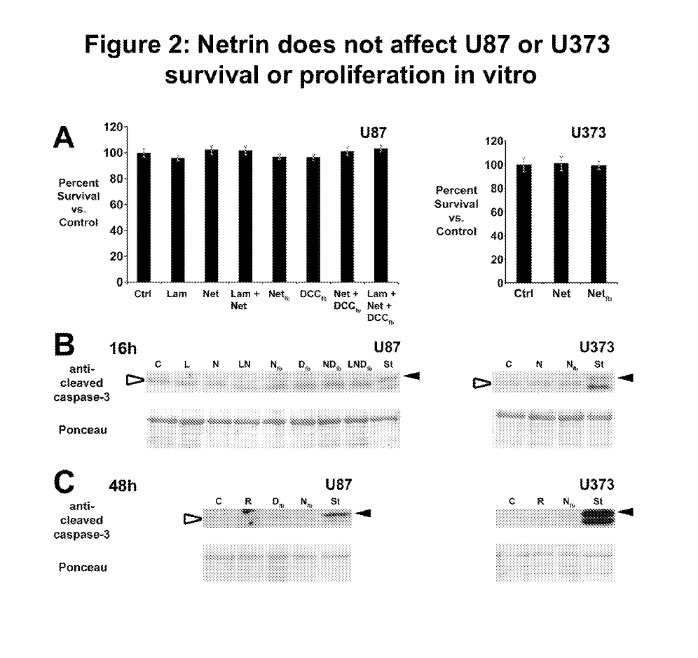

DCC and UNC5 homologues have been proposed to function as dependence receptors, activating apoptosis in the absence of netrin-1 (Mehlen. P. and L. Mazelin, 2003, Biol. Cell 95:425-436). This raises the possibility that the effects described above may be due to an influence on cell survival and not motility. Thus, we examined the consequences of manipulating netrin function on the survival of U87, U343 and U373 cells. No significant change in cell number (FIG. 2A), or activation of caspase-3, a sensitive indicator of apoptosis (FIG. 2B), was detected following 16 his treatment with exogenous netrin-1, laminin-1, or both; nor following disruption of netrin or DCC function using blocking antibodies. Further testing, by blocking netrin and DCC function for 48 hrs, again detected no increase in caspase-3 activation (FIG. 2C). In contrast, staurosporine, applied as a positive control, activated caspase-3 and caused extensive cell death (FIGS. 2B, C). These findings are consistent with previous analyses of glial precursor cells, indicating that netrin-1 and DCC do not regulate oligodendroctye precursor survival either in vitro or in vivo (Jarjour, A. A., et al., 2003, J. Neurosci. 23:3735-3744; Tsai, H. H., et al., 2006, J. Neurosci. 26:1913-1922), and they support the conclusion that the results found using the transfilter assays reflect changes in cell migration and not effects on cell survival or proliferation.

### Example 9

### Endogenous Netrin Promotes the Maturation of Focal Complexes into Focal Adhesions

Cell migration requires the formation of transient adhesive contacts with the extracellular matrix (ECM). Initial contacts occur at the leading edge of lamellipodia where integrins bind ECM ligands and recruit proteins such as vinculin and paxillin to form immature adhesive contacts (focal complexes, or FC) (reviewed by Wozniak, et al., 2004, Biochim. Biophys. Acta 1692:103-119). The transition from FC to focal adhesions (FA) is marked by consolidation of the adhesive contact, an increase in size, and the recruitment of additional proteins including tensin and zyxin (Zaidel-Bar. R., et al., 2003, J. Cell Sci. 116:4605-4613).

The effect of disrupting netrin function on adhesive complex formation in glioblastoma cells was investigated by examining the distribution of paxillin, which is present in both FAs and FCs, and zyxin, which is present in FAs but not FCs. The influence of netrin on FC formation was quantified by subtracting the distribution of zyxin immunoreactivity from paxillin immunoreactivity to create images representing regions of paxillin, but not zyxin localization. Using the ‘paxillin minus zyxin’ images, the density of FCs present in each lamellipodium was calculated. Exposure of U87 cells to a control preimmune antibody (RbIgG). DCC_{FB}, or netrin-1, resulted in no change relative to control. In contrast, application of Netrin_{FB} resulted in increased FC density. A similar increase was observed when netrin function was inhibited in U373 cells, but not U343 cells, in which FC density was high in all conditions examined (data not shown).

To determine if inhibiting endogenous netrin function influences FA density, images depicting regions of paxillin and zyxin colocalization were generated. From the ‘paxillin and zyxin’ images, the density of FAs in each lamellipod was calculated. In U87 and U373 cells, addition of netrin function-blocking antibody resulted in decreased FA density. In all other conditions analyzed for U87 and U373 cells and in all conditions analyzed for U343 cells, no change in FA density was observed. Notably, the increase in FC density and corresponding decrease in FAs correlates precisely with the changes in motility evoked by disrupting netrin function and measured using the microchemotaxis assay (FIG. 3). These data are consistent with a mechanism in which netrin promotes the maturation of FCs into FAs, and wherein these adhesive structures act to restrain cell movement.

### Example 10

### Netrin and Netrin Receptors are Localized to Focal Adhesions, but not Focal Complexes

We then investigated the possibility that netrin and netrin receptors might be localized to FCs or FAs and thereby directly influence their maturation. U87. U343, and U373 cells were labeled with the following antibodies: mouse monoclonal anti-paxillin and one of goat polyclonal anti-DCC, or rabbit polyclonal antibodies against netrin or unc5 homologues. U87 cells were also labeled with goat polyclonal anti-DCC and rabbit polyclonal anti-zyxin (FIG. 4). In U87 cells netrin (FIG. 4A-C), DCC (FIG. 4M-O) and UNC5 homologue (FIG. 4G-I) immunoreactivity colocalized with large paxillin-positive foci characteristic of FAs (white arrowhead), but not smaller paxillin-positive structures characteristic of FCs (black arrowhead). In U343 and U373 cells, that lack DCC expression, netrin (FIG. 4D-F, P-R) and UNC5 homologue (FIG. 4J-L, V-X) immunoreactivity was similarly localized to FAs but not FCs. Consistent with localization to FAs. DCC and zyxin immunoreactivity colocalized in U87 cells (FIG. 4S-U). Colocalization with markers of FAs is consistent with netrins and netrin receptors regulating cell-substrate adhesion and motility.

### Example 11

### Netrin-1 VI-V Peptide Inhibits Cell Migration, but does not Promote Migration

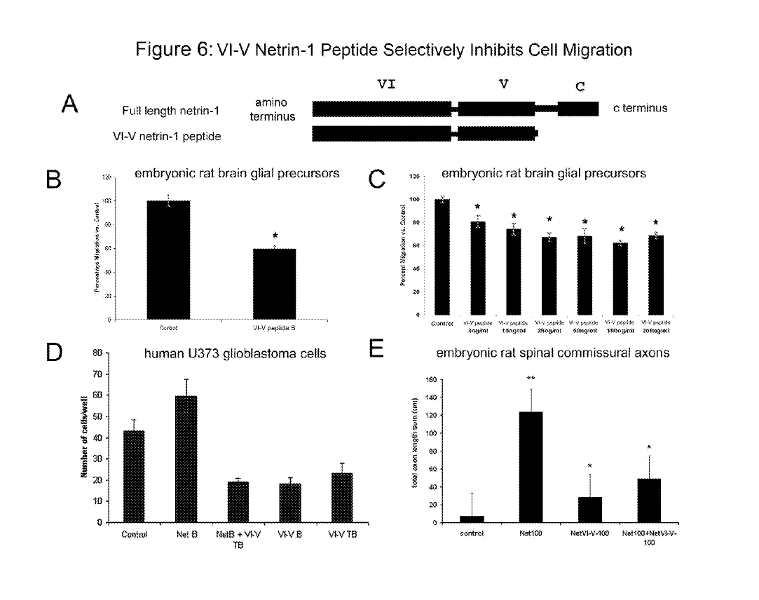

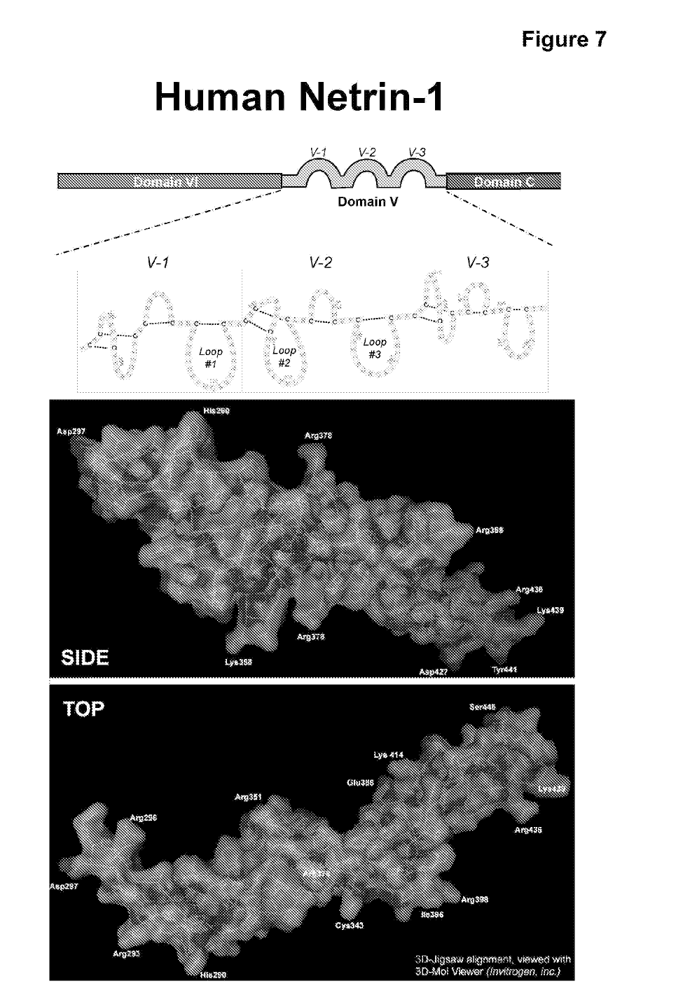

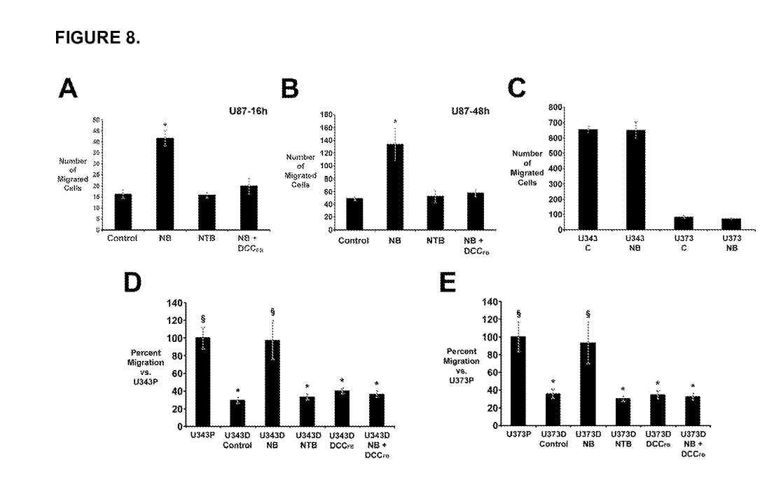

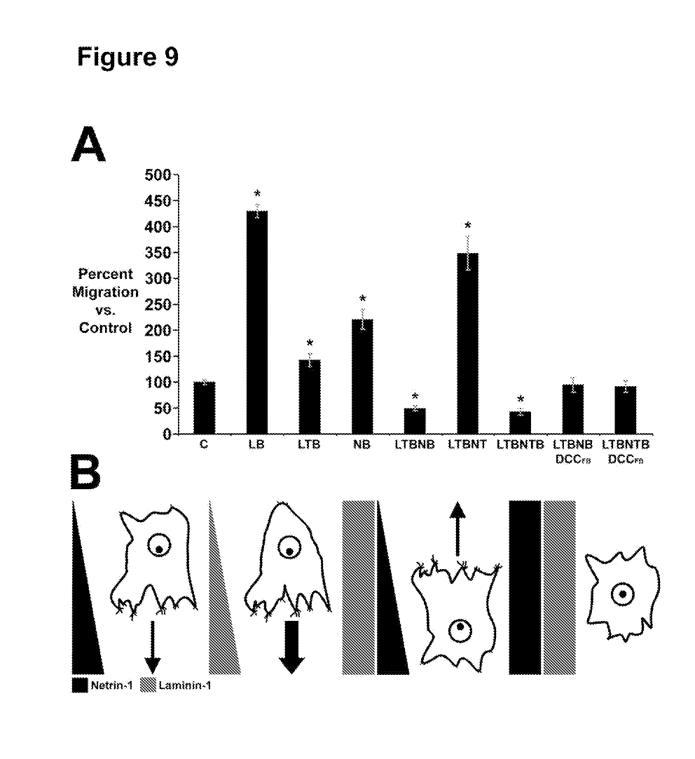

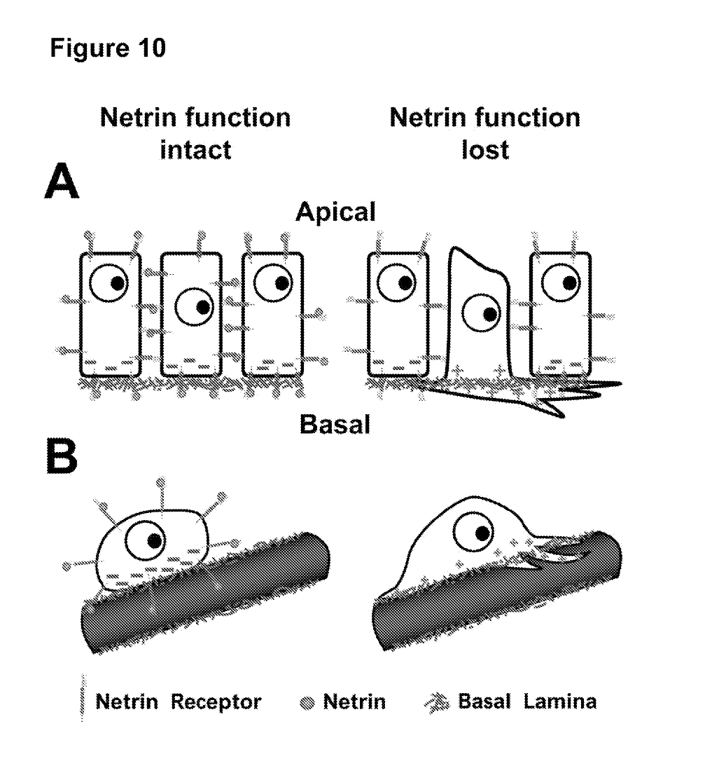

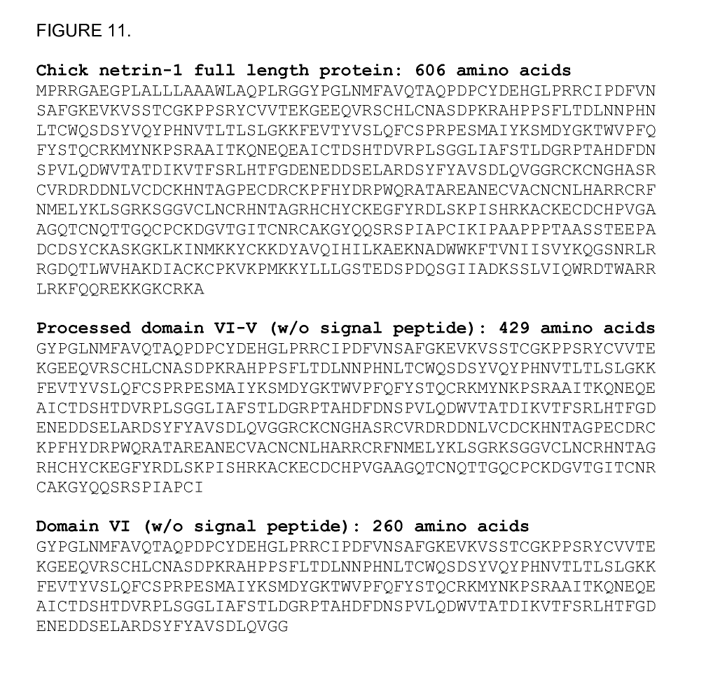

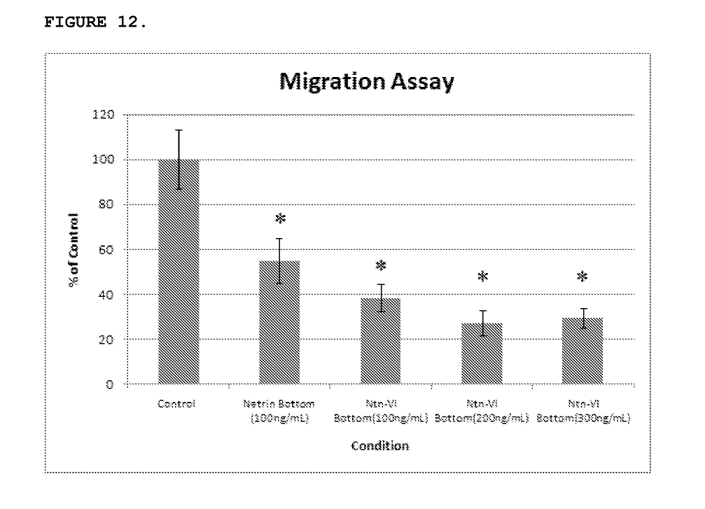

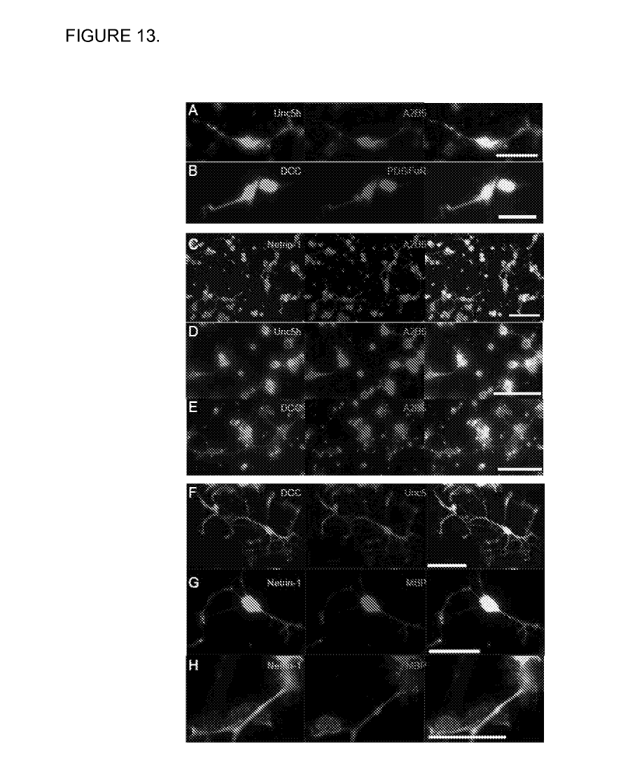

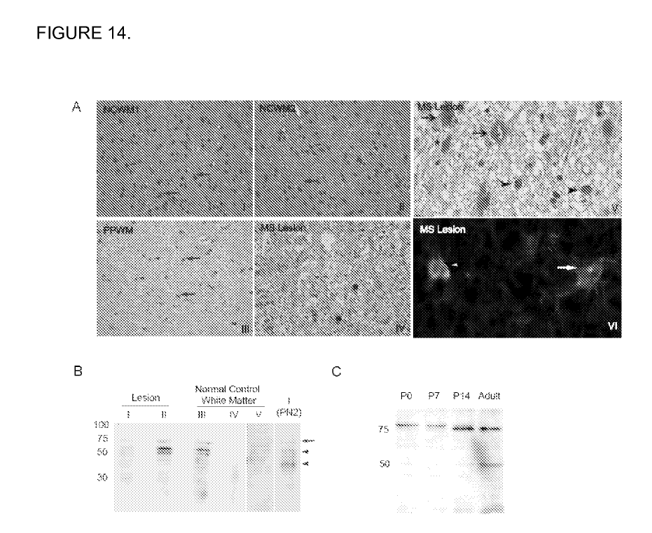

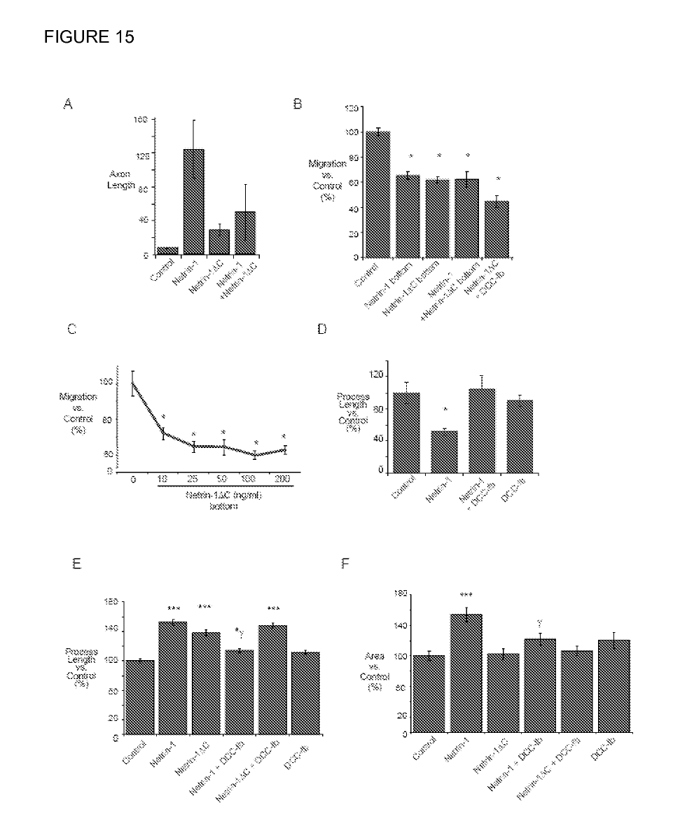

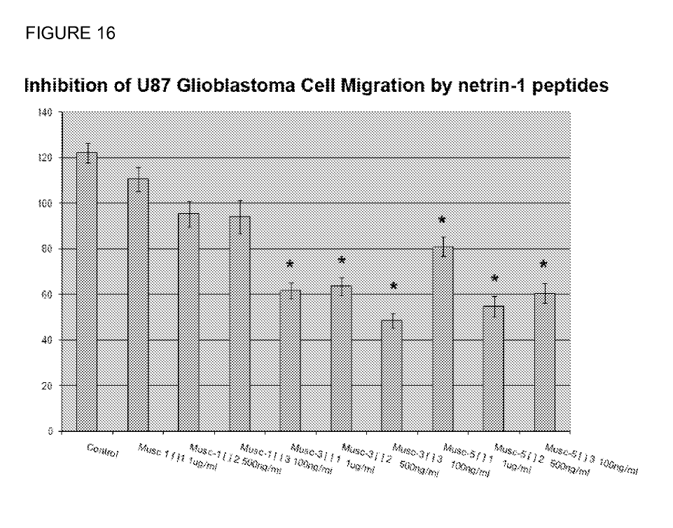

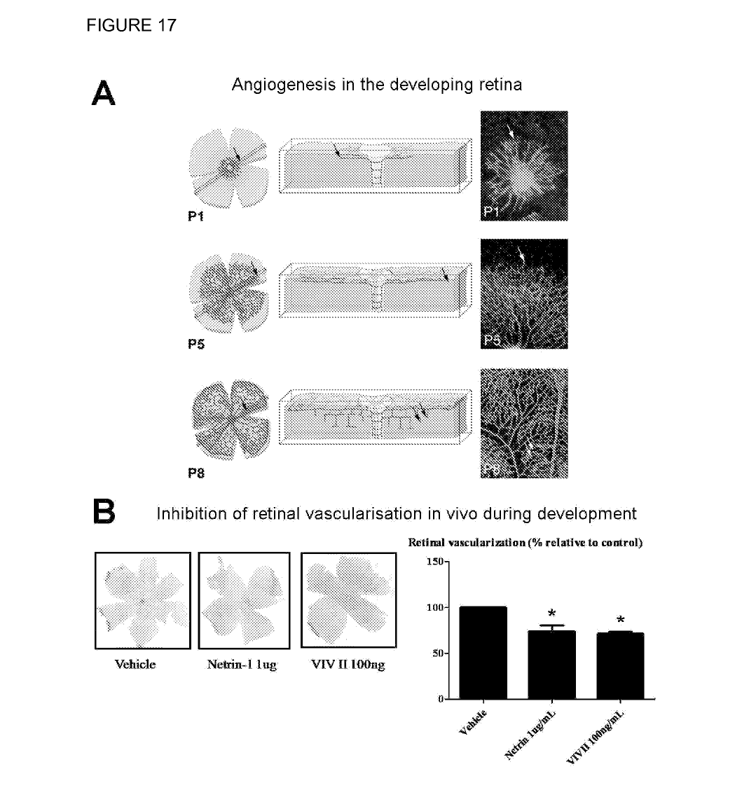

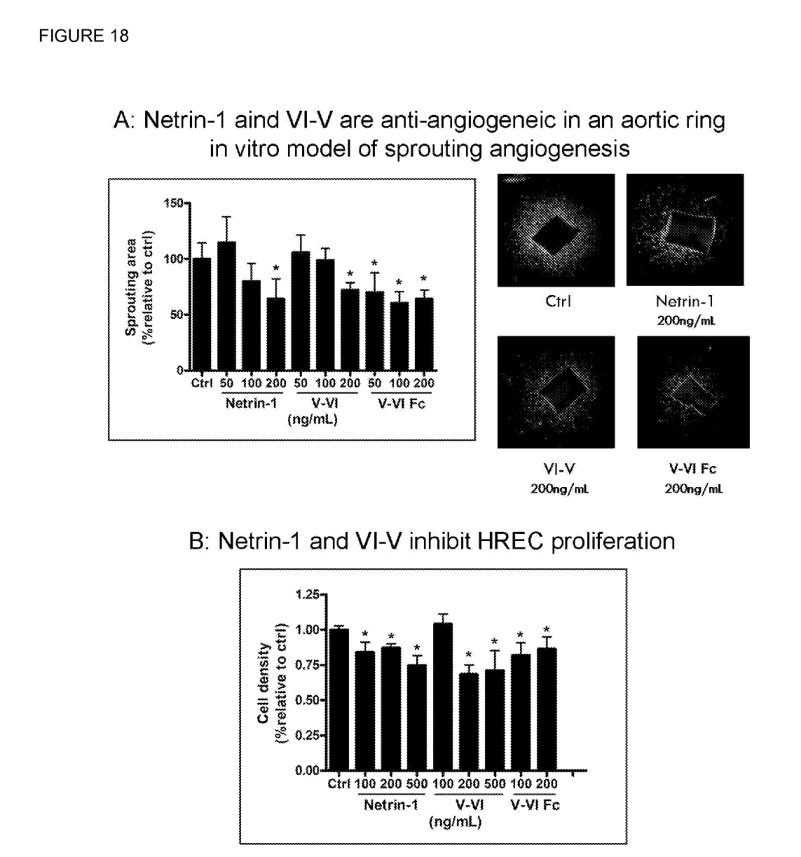

We tested whether a deletion of netrin-1 lacking domain C, netrin-1 VI-V peptide (also referred to as netrin-1 AC peptide), inhibits glial precursor and glioblastoma cell migration, but has lost the capacity of full-length netrin-1 to promote migration (FIG. 6). Application of the netrin-1 VI-V peptide either as a gradient in the transfilter migration assay (B: bottom well alone), or uniformly at an equal concentration to the top and bottom chambers of the migration assay (TB: top and bottom) significantly reduced the number of rat glial precursor cells (FIG. 6B,C), or human glioblastoma cells (FIG. 6D) that migrated across the filter. In contrast, the VI-V peptide did not evoke outgrowth of commissural axons from an explant of embryonic rat spinal cord (FIG. 6E), a standard assay used to demonstrate DCC dependent axon outgrowth evoked by netrin-1 (Kennedy, T. E., et al., 1994. Cell 78:425-435). In contrast, when applied together the VI-V peptide appears to antagonize the capacity of full-length netrin-1 to promote axon outgrowth (FIG. 6E). Our results show that netrin-1 VI-V selectively inhibits cell migration by activating UNC5 homologue netrin receptors, without activating DCC. These surprising and unexpected results represent the first demonstration that a derivative of netrin could selectively inhibit cell migration, in contrast to full-length netrin which is bifunctional. The VI-V fragment and derivatives therefore which retain function as selective inhibitors of cell migration are therefore more amenable for use as therapeutic agents.

### Example 12

### Netrin-1 Domain VI is Sufficient to Repel Glial Precursor Cell Migration

We tested how the migration of glial precursor cells (oligodendrocyte precursor cells isolated from newborn rat brain) was affected in response to either full length purified recombinant netrin-1, or purified recombinant protein chimera composed of netrin-1 domain VI linked to a human antibody Fc domain (FIG. 12). For the netrin-1 domain VI-Fc chimera, we cloned domain VI into the pFUSE-hFc1 vector (InvivoGen, San Diego, Calif.), which contains a human IgG₂ Fc. Migration was tested in a Boyden chamber as illustrated in FIG. 3C. Cells were plated on the top side of a porous membrane. In the control condition, the count corresponds to the number of cells that spontaneously migrated from the top side of the membrane to the bottom side of the membrane. In the netrin bottom (100 ug/ml) condition, full length netrin-1 protein placed in the bottom portion of the chamber repelled the migration of glial precursor cells across the membrane, resulting in fewer cells migrating than control. The leftmost 3 histogram bars illustrate the number of cells migrating in response to netrin-1 domain VI-Fc chimera placed in the bottom chamber. A dose response was carried out, placing either 100 ug/ml, 200 ug/ml, or 300 ug/ml in the bottom chamber, as indicated. The reduced number of cells migrating across the membrane indicates that netrin-1 domain VI is sufficient to repel glial precursor cell migration. Four independent replicates of each condition were used: n=4. Statistical comparison utilized an ANOVA test with a Tukey HSD correction; * indicates P<0.001 compared to control.

It is noted that this finding that domain VI is sufficient to inhibit migration was surprising and unexpected. Previously, genetic analyses provided evidence that this function of netrin-1 would reside in domain V (see FIG. 7). Our results demonstrating that domain VI is sufficient for inhibition suggest that the previously-characterized domain V mutation is more likely to abrogate netrin function in a general way, and is not the key site for netrin-1 inhibitory function.

### Example 13

### Peptides Corresponding to Sequence in Human Netrin-1 Inhibit the Migration of Human U87 Glioblastoma Cells

Peptides corresponding to three different highly conserved sequences found in domains VI and V of netrin-1 were synthesized, as follows: musc-1: KPFHYDRPWQRATAREANEC; musc-3: IYKSMDYGRTVVVPF; and musc-5: RFNMELYKLSGRKSGGVC. The peptides were added in media to the bottom well of Boyden chamber transfilter micochemotaxis assays, as illustrated in FIG. 3C. Cells (U87 human glioblastoma cell line) were plated in the top chamber and the number of cells migrating across the porous membrane, from the top side to the bottom side, were counted. The control condition reveals the spontaneous migration of the cells, moving in the absence of any added cue. Peptides were added to the bottom well at concentrations of (1) 1 ug/ml, (2) 500 ng/ml, and (3) 100 ng/ml.; *indicates P<0.05 compared to control (FIG. 16). The results provide evidence that the peptides can inhibit U87 cell migration.

### Example 14

### Netrin-1 and Proteolytic Fragments of Netrin-1 are Present in Multiple Sclerosis Lesions and Induce Distinct Morphological Responses in Human Oligodendroglia

Netrin-1 is known to be a chemotropic guidance cue that plays critical roles in oligodendrocyte precursor cell (OPC) migration, oligodendrocyte (OLG) maturation and myelin maintenance. We determined that human fetal and adult OPCs, and human adult OLGs, express the netrin-1 receptors DCC and the Unc5 homologs in vitro (FIG. 13). In addition, human adult OPCs and OLGs showed autocrine expression of netrin-1. We detected the netrin-1 immunoreactivity associated with OLGs and astrocytes in sections of normal white matter, and also observed netrin-1 immunoreactivity in OLGs, astrocytes, macrophages and the extracellular matrix (ECM) in MS lesions (FIG. 14). Biochemical analysis indicated that in addition to the full-length protein, proteolytic fragments of netrin-1 were present and abundant in MS lesions, and could also be detected in adult rodent CNS and normal control white matter (NCWM). These fragments lack the globular C-domain of full-length netrin-1. Human oligodendroglia responded to both netrin-1 and a truncated netrin-1 construct lacking the C domain (FIG. 15). Netrin-1 and the truncated construct repelled rodent OPCs and increased process extension in adult human OPCs, but only full length netrin-1 promoted oligodendrocyte membrane area and axon extension. These results indicated that the proteolytic fragments of netrin-1 present in MS lesions may not be as effective as full length netrin-1 in inducing the morphological changes in oligodendroglial that lead to remyelination, and suggest that detecting the presence of the truncated netrin-1 may be useful for diagnosis and prognosis of MS.

We first investigated expression of netrin-1 receptors and netrin-1 by human oligodendroglia. Rodent OPCs and OLGs express the netrin-1 receptors DCC and the Unc5 homologs, and netrin-1 immunoreactivity is observed in myelin-like membrane sheets of oligodendrocytes (Rajasekharan S. et al., 2009. Development 136:415-426). We analyzed human oligodendroglia for the expression of netrin-1 receptors and netrin-1 in vitro. Human fetal OPCs (HF-OPCs) were immunopositive for A2B5 or the platelet derived growth factor receptor a (PDGFαR), human adult OPCs (HA-OPCs) expressed A2B5 and human adult oligodendrocytes (HA-OLGs) expressed myelin basic protein (MBP) or myelin associated glyocoprotein (MAG) (FIG. 13A-H, red). We detected the presence of the netrin-1 receptors DCC and the Unc5 homologs in all three human oligodendrocyte lineage cells types, indicating that these cells share similar receptor expression profiles as their rodent counterparts (FIG. 13A, B, D-F). In addition, we observed netrin-1 immunoreactivity in human adult OPCs (FIG. 13C, green) and OLGs (FIG. 13F, green). Netrin-1 protein was preferentially localized to the extending processes of HA-OPCs and HA-OLGs, but also was found in the myelin-like membrane sheets of HA-OLGs (FIG. 13C, G, H, green).

During development, oligodendrocytes express autocrine netrin-1 after the initiation of myelination, and in the adult rodent CNS (Rajasekharan S. et al., 2009. Development 136:415-426), netrin-1 is associated with non-compact myelin membranes (Manitt, C., et al., 2001, J. Neurosci. 21:3911-3922). We next characterized the expression pattern of netrin-1 in the human CNS. We detected netrin-1 immunoreactivity associated with oligodendrocytes in sections of normal control white matter (NCWM) (FIG. 14Ai, ii, arrowheads). In contrast to the rodent CNS (Manitt, C., et al., 2001, J. Neurosci. 21:3911-3922), we were also able to detect netrin-1 expression in astrocytes (FIG. 14Ai, ii, arrows). Mature, myelinating OLGs in the human adult CNS, like their rodent counterparts, are immunoreactive for netrin-1. We next asked whether netrin-1 is present in demyelinated lesions, and whether it is associated with the same cell types seen in NCWM. Sections of tissue from three MS cases were analyzed (Table 1) for netrin-1 immunoreactivity in lesions, periplaque white matter (PPWM) normal appearing white matter (NAWM). As was the case with NCWM, netrin-1 immunostaining revealed a distribution of netrin-1 protein associated with oligodendrocytes and astrocytes (FIG. 14A, iii-yl, arrowheads, arrows). However, we also detected netrin-1 immunoreactivity in macrophages (FIG. 14A, iv, asterisk), and there was an increased diffuse stain associated with the ECM. These results suggest that demyelination appears to release netrin-1 into the lesion environment, where it is associated with the ECM, and encountered by phagocytic macrophages. Our results indicate that Netrin-1 is detected in normal white matter and MS lesions.

Biochemical analysis of post mortem tissue samples in normal and MS cases was conducted to confirm the presence of netrin-1 in these tissues (Table 1). Western blot analyses of protein lysates from these samples confirmed that netrin-1 protein is present in NCWM and MS lesions (FIG. 14B). Surprisingly, in addition to full length 75 KDa netrin-1 immunoreactive band (FIG. 14B, arrow), we also detected the presence of shorter netrin-1 immunoreactive bands ranging between 50 and 30 KDa (FIG. 14B, arrowheads). To confirm the specificity of this immunoreactive pattern we performed western blot analyses with a second, well characterized netrin-1 antibody, PN2 (FIG. 14B, PN2) (Manitt, C., et al., 2001, J. Neurosci. 21:3911-3922). From the immunohistochemical and biochemical data we conclude that full length and fragmented forms of netrin-1 are present in the adult CNS. Proteolytic fragments of netrin-1 are found in both normal and MS tissue.

TABLE 1
Characteristics of post-mortem human brain material

| | | | | | | Time |
|---|---|---|---|---|---|---|
| | | | | | | between |
| | | | | | | Death and |
| | | | | | | Sample |
| | Sample | Age | | | | Acquisition |
| Label | Type | (yrs) | Case | M/F | Cause of Death | (hrs) |
| I | Lesion | 62 | MS | M | Septicemia and | 28 |
| | | | | | Broncho- | |
| | | | | | pneumonia | |
| II | Lesion | 55 | MS | F | Broncho- | 9 |
| | | | | | pneumonia | |
| III | NCWM | 75 | No CNS | F | Cardiac Arrest | 7 |
| | | | Disease | | | |
| IV | NCWM | 34 | No CNS | M | Myocardial | 21 |
| | | | Disease | | Infarction | |
| V | NCWM | 78 | No CNS | F | Bronchopneu- | 20 |
| | | | Disease | | monia, chronic | |
| | | | | | heart disease, | |
| | | | | | thyroid disease | |

The shorter netrin-1 fragment found in adult CNS tissue was characterized further. We immunoprecipitated netrin-1 from a rat brain homogenate and isolated the shorter fragment by gel electrophoresis. The shorter band was cut out and analysed by mass spectrometry. Peptide sequences obtained corresponded to the VI and V domains of full length netrin-1, but not to the C domain (data not shown). Furthermore, the netrin-specific PN2 antibody was raised against a peptide located in the VI and V domain of netrin-1, and there is evidence suggesting that MAB1109 recognizes this region (N. Marcal and T. E. Kennedy, unpublished data). We conclude that the shorter anti-netrin-1 immunoreactive fragments present in MS lesions correspond to truncated forms of full length netrin-1, lacking the C terminal domain. Thus fragments of netrin-1 in adult CNS correspond to the VI-V domain of full length netrin-1.

Netrin-1 is a chemoattractant for DCC expressing spinal commissural neurons, and promotes outgrowth of commissural axons from dorsal spinal cord explants (FIG. 15A, Netrin-1, 100 ng/ml; Kennedy et al., 1994). It is also known that the VI-V domain of netrin-1 is sufficient to bind DCC (Keino-Masu K. et al., 1996. Cell 87:175-185). Therefore we generated a recombinant netrin-1 construct lacking the C domain (netrin-1ΔC) to test the functional activity of the fragment of netrin-1 observed in the adult CNS. Netrin-1ΔC (100 ng/ml) did not promote commissural axon outgrowth from dorsal spinal cord explants (FIG. 15A, Netrin-1ΔC), but blocked the ability of full length netrin-1 to induce axon outgrowth (FIG. 15A Netrin-1 +Netrin-1ΔC), possibly by competing for DCC binding sites. These results show that a recombinant construct of netrin-1 lacking the C domain repels OPCs but does not attract commissural axons.

Netrin-1 induces the DCC-dependent chemorepulsion of migrating OPCs in vitro (Jarjour. A. A., et al., 2003, J. Neurosci. 23:3735-3744; Tsai, H. H., et al., 2003. Development 130:2095-2105). Addition of netrin-1 (100 ng/ml) to the bottom of a well in a microchemotaxis assay reduces the number of OPCs migrating through a transwell filter compared to control (FIG. 15B, Netrin-1). Similarly, netrin-1ΔC also repels migrating OPCs (FIG. 15B, Netrin-1ΔC), but addition of a DCC function blocking antibody (DCC-fb) did not disrupt this netrin-1ΔC dependent chemorepulsion (FIG. 15B, Netrin-1ΔC +DCC-fb). A dose-response analysis determined that a concentration of 100 ng/ml of netrin-1ΔC provided maximal repulsion (FIG. 15C). Taken together, these results indicate that a truncated form of netrin-1 does not induce outgrowth in commissural axon, but retains the ability to repel OPCs in a DCC-independent manner.

The expression of netrin-1 in MS lesions led us to ask whether netrin-1 induces morphological changes in human oligodendroglia. We first investigated whether netrin-1 is able to recapitulate its developmental effects in human fetal cells. During rodent development, netrin-1 repels OPCs by inducing a DCC-dependent decrease in process extension (Jarjour. A. A., et al., 2003, J. Neurosci. 23:3735-3744; Tsai, H. H. et al., 2003. Development 130:2095-2105). Addition of recombinant netrin-1 to cultures of HF-OPCs induced a significant decrease in process length (FIG. 15D, Netrin-1), and this decrease in process length was disrupted by the addition of a DCC function-blocking antibody (FIG. 15D, Netrin-1 +DCCfb). These results show that human fetal OPCs and rodent OPCs exhibit similar morphological responses to netrin-1. The results show also that Netrin-1 induces distinct morphological changes in HF-OPCs, HA-OPCs and HA-OLGs.

Human adult OPCs are recruited to sites of demyelination, where they differentiate into adult oligodendrocytes, capable of remyelination. We investigated therefore whether full length and truncated forms of netrin-1 present in MS lesions can induce morphological changes in HA-OPCs. Addition of netrin-1 to cultures of HA-OPCs resulted in an increase in process length (FIG. 15E, Netrin-1) and this was blocked by a DCC function blocking antibody (FIG. 15E, Netrin-1 +DCC-fb). This result indicates that netrin-1 induces distinct responses in fetal and adult OPCs. Netrin-1ΔC also increased the length of HA-OPC processes (FIG. 15E, Netrin-1 AC); however, a DCC function blocking antibody did not inhibit the effect of netrin-1ΔC in human adult OPCs (FIG. 15E, Netrin-1ΔC +DCC-fb), as is the case in the netrin-1 AC dependent repulsion of rodent OPCs (FIG. 15B, Netrin-1 AC +DCC-fb). Thus, netrin-1 and netrin-1ΔC induce distinct morphological changes HA-OPCs and HA-OLGs.

In later stages of oligodendrocyte development, netrin-1 induces an increase in process extension and branching in mature rodent OLGs (Rajasekharan G. et al., 2009. Development 136:415-426). Human OLGs are morphologically less complex than their rodent counterparts, so we measured the surface area of the cells, which includes both processes and myelin-like membrane sheets. Netrin-1 (100 ng/ml) induced an increase in the surface area of HA-OLGs (FIG. 15F, Netrin-1), and this was blocked by the addition of DCC-fb (FIG. 15F, Netrin +DCC-fb). However, netrin-1ΔC (100 ng/ml) did not induce a morphological change in HA-OLGs (FIG. 15F, Netrin-1ΔC.). Thus, while full length netrin-1 may promote a morphological change in OLGs associated with the initiation of myelination, truncated forms of netrin-1 lose the ability to promote oligodendrocyte maturation.

### Example 15

### Netrin-1 and the Netrin-1 Vi-V Peptide Inhibit Angiogenesis in the Retina During Development

The vascular plexus of the rodent retina largely forms postnatally during the first week of life. It can therefore be modulated by compounds injected into the vitreous humor of the eye during the initial post-natal period (Sennlaub. F., et al. Circulation 108, 198-204 (2003)). In order to determine whether netrin-1 and the netrin-1 VI-V peptide inhibit angiogenesis in the retina during development, Netrin-1 and netrin-1 VI-V peptide were injected intravitreously at an estimated intraocular concentration of 100 ng/ml to 1 ug/ml on post-natal day 1 (P1). Retinal vascularisation was assessed at P4 and found to be substantially diminished (FIG. 17). The results indicate inhibition of retinal vascularization in vivo during development.

### Example 16

### Netrin-1 and the Netrin-1 VI-V Peptide Inhibit Vascular Sprouting Ex Vivo

Sections of aortae were dissected from adult C57BL6 mice, then cut into 1 mm thick rings and embedded in growth factor-reduced Matrigel (BD Biosciences) in 24-well tissue culture plates. Microvascular sprouting growing out of the rings was quantified by measuring the area covered by outgrowth of the aortic ring using ImagePro Plus 4.5 (Media Cybernetics, Silver Spring, Md.). Aortic rings were treated with netrin-1 or the netrin-1 VI-V peptide, either VI-V alone, or a VI-V-Fc protein chimera. Significantly reduced vascular growth was found in all three treatment groups (FIG. 18A). The results indicate that Netrin-1 and the netrin-1 VI-V peptide are anti-angiogenic in an aortic ring in vitro model of sprouting angiogenesis.

We also showed that Netrin-1 and the netrin-1 VI-V peptide inhibit endothelial cell proliferation. Human retinal endothelial cells (HRECs) were obtained from Cell Systems (Kirkland, USA) and used from passage 2-7. Cell number was determined using the MTT (3-(4,5-dimethylthiazol-2-yl)-2,5-diphenyltetrazolium bromide) assay as described (Brault, S., et al., Am J Physiol Regul Integr Comp Physiol 292, R1174-1183 (2007)). Netrin-1 and the netrin VI-V peptide, either VI-V alone, or a VI-V-Fc protein chimera were introduced to media upon seeding and cell viability assessed after 36 hours (FIG. 18B). The results indicate that Netrin-1 and the netrin-1 VI-V peptide inhibit RBEC proliferation.

### Materials and Methods

### Cells and Cell Culture

Human glioblastoma cell lines, U87, U343, U373 (ATCC, Rockville, Md.) and astrocytes isolated from newborn mouse brain were grown as monolayer cultures in DMEM (Invitrogen, Burlington, ON), 10% heat-inactivated fetal bovine serum (FBS, Invitrogen), glutamax-1 (Invitrogen) and penicillin/streptomycin.

Antibodies, Conditioned media, cell lysates, western blotting, and PCR

Antibodies against the following were used: cleaved caspase-3 (Asp175, mouse, Cell Signaling Technology, Beverly, Mass.); DCC (DCC_{IN}, mouse, G97-449; BD Biosciences Pharmingen, San Jose, Calif.; DCC_{GT}, goat, A-20; Santa Cruz Biotechnology, Santa Cruz, Calif.; function-blocking, DCC_{FB}, mouse, AF5; Calbiochem, La Jolla, Calif.); netrin-1 and 3 (PN2, rabbit, Manitt et al., 2001; netrin function-blocking (Netrin_{FB}. PN3, rabbit, Manitt et al., 2001; neogenin (rabbit, Santa Cruz Biotechnology); paxillin (mouse, BD Biosciences Pharmingen); pan-unc5h (rabbit, Tong, J., et al., 2001, J. Biol. Chem. 276:40917-40925, provided by Dr. Tony Pawson, Mount Sinai Hospital, Toronto, ON; preimmune rabbit IgGs (RbIgG; Invitrogen); and zyxin (rabbit, Abcam, Cambridge, Mass.).

Cultures were grown to 80% confluence and conditioned media collected following 48 hrs in serum-free DMEM. For lysates, cells were grown to 80% confluence, rinsed with PBS and lysed in 1 ml of hot sample buffer (60 mM Tris/HCl, pH 6.8, 2% SDS, 10% glycerol, 100 mM DTT). For western blot analysis of cleaved caspase-3, cells were cultured at a density of 120,000 cells/well in a 12-well tissue culture plate. Nitrocellulose immunoblots were probed with DCC_{IN} (0.5 μg/ml), PN2 anti-netrin (4 μg/ml), anti-cleaved caspase-3 (1:1000), or anti-neogenin (0.4 μg/ml). After washing, membranes were incubated with HRP-coupled secondary antibodies and immunoreactivity visualized using chemiluminescence (NEN, MA).

PCR was carried out using standard methods.

### Transfilter Chemotaxis Assay

Cells were plated at a density of 4×105 cells/ml on polycarbonate transwell culture inserts (6.5 mm diameter with 8 μm pore size. Corning). 100 μl of cell suspension was used per filter, and the filters placed in the wells of a 24-well plate over 600 μl of medium. DMEM with 0.2% BSA, 100 U/ml penicillin, 100 μg/ml streptomycin, and 2 mM glutamax was the base medium used for all assay conditions. Following migration, cells on the upper side of the filter were scraped off, and the cells attached to the lower side of the filter fixed with 4% paraformaldehyde (PFA)/0.1% glutaraldehyde (30 min, 4° C.). Filters were rinsed with PBS, cell nuclei stained with Hoechst dye. Four transwells were used per condition. Four images of each filter were captured using a 10× objective and nuclei counted using Northern Eclipse software (Empix Imaging, TO). Where pooled results are presented, the value ‘percent migration vs control’ for a given trial represents the number of cells migrated in that condition expressed as a percentage of the mean number of cells migrating in control conditions. Recombinant netrin-1 protein was purified as described (Shekarabi. M., et al., 2005, J. Neurosci. 25:3132-3141) and used at a concentration of 100 ng/ml. Laminin-1 was used at 10 μg/ml (BD Biosciences, Bedford, Mass.). NetFB and rabbit preimmune IgG (used as a control) were added at a concentration of 25 μg/ml. DCCFB was added at a concentration of 10 μg/ml.

### Plasmids and Transfection

U343 and U373 cells were transfected using lipofectamine (Invitrogen) with expression constructs encoding either green fluorescent protein (GFP) alone or DCC tagged at its C-terminus with GFP (Shekarabi, M. and T. E. Kennedy, 2002, Mol. Cell Neurosci. 19:1-17). Seventy-two hrs after transfection, the medium was changed to selection medium containing Geneticin (Invitrogen).

### Confocal Image Analysis

10⁴ cells were plated per well in chamber slides (Fisher) coated with 20 μg/ml poly-D-lysine (Sigma) at 4° C. overnight, washed with Hanks buffered salt solution (Invitrogen) and allowed to dry. Cells were fixed in 4% PFA, 4% sucrose in PBS, and permeabilized with 0.25% Triton X-100 in PBS. Blocking was performed in 3% heat-inactivated normal goat serum, 2% BSA, and 0.125% Triton X-100 in PBS. Cells were then incubated with anti-paxillin and anti-zyxin (FIG. 5), anti-paxillin and one of anti-netrin PN2, anti-UNC5, or anti-DCC_{GT}, or anti-zyxin and anti-DCC_{GT} (FIG. 4) diluted in blocking solution. Primary antibodies were detected with secondary antibodies coupled to Alexa 546 or Alexa 488 (Molecular Probes).

For imaging adhesive complexes, single confocal optical slices through the base of lamellipodia were collected. Identical settings were used for each condition examined for a given cell line. The outermost region of individual lamellipodial protrusions (excluding regions of paxillin or zyxin immunoreactivity contiguous with adhesive structures in the cell body) was outlined using Image J software (Rasband, W. S. ImageJ. U.S. National Institutes of Health, Bethesda, Md., USA, http://rsb.info.nih.gov/ij/, 1997-2006). Mask images were then generated representing either the regions staining with both paxillin and zyxin (using an ‘AND’ function) or representing the difference between the paxillin and zyxin images (the zyxin signal subtracted from the paxillin image). Images were adjusted to eliminate signal below a minimum value that was held constant across all images for each cell line. Signal in the ‘AND’ image corresponds to the area of each lamellipodium occupied by mature FAs that contain both paxillin and zyxin. To quantify FCs, the subtracted image representing paxillin staining without zyxin was filtered to exclude structures smaller than 3 pixels or larger than 40 pixels. The number of individual adhesions was then counted and the density of adhesions within each lamellipodium calculated. Netrin-1 was used at 100 ng/ml, NetFB and rabbit preimmune IgG (as a control) at 25 μg/ml, and DCCFB at 10 μg/ml.

### Analysis of Cell Number and Apoptosis

To investigate changes in cell survival or proliferation, cells were plated at a density of 30,000 cells per well in 8-well chamber slides (Fisher), allowed to settle for 2 hrs, treated as described for 16 hrs, fixed and stained with Alexa-488 phalloidin and Hoechst, and the number of live cells per 20× field counted. To measure levels of apoptosis in these cultures, 120,000 cells were cultured in each well of a 12-well tissue culture plate, allowed to settle for 2 hrs, and treated as described for either 16 or 48 hrs. In all cases, the base medium used was DMEM with 2% FBS, penicillin/streptomycin, and glutamax-1.

### Animals

Sprague Dawley rat pups were obtained from Charles River Canada (Quebec, Canada). All procedures were performed in accordance with the Canadian Council on Animal Care guidelines for the use of animals in research.

### Rodent OPC Culture

OPCs were derived from mixed glial cultures from the cerebral cortices of postnatal day 0 rat pups and were grown in oligodendrocyte defined medium (OLDEM) as described previously (Armstrong R C, 1998, Methods 16:282-292; Jarjour, A. A., et al., 2003, J. Neurosci. 23:3735-3744), with 0.1% fetal bovine serum (FBS) to initiate differentiation of mature oligodendrocytes,

### Human Fetal OPC Culture

Human fetal CNS tissue obtained from 19- to 23-week-old embryos was provided by the Human Fetal Tissue Repository (Albert Einstein College of Medicine, Bronx, N.Y.). The studies were approved by their and our institutional review boards. Isolation of human fetal progenitor cells was performed as previously described (Ruffini F. et al., 2004, Am. J. Pathol. 165:2167-2175; Miron V E, et al., 2007. Glia 55:130-143). Briefly, diced brain tissue was incubated with 0.25% trypsin (Invitrogen) and 25 μg/mL DNase I (Roche, Laval, QC) in a 37° C. water bath for 30 min, and washed with phosphate-buffered saline (PBS) through a 132-μm nylon mesh (Industrial Fabrics, Minneapolis, Minn.). Cells were incubated on ice with anti-A2B5 μM antibody, washed with MACS buffer (2 mM/L ethylenediaminetetraacetic acid, 0.5% fetal calf serum in PBS), and incubated with the microbead-conjugated rat anti-mouse IgM antibody (Miltenyi Biotech, Auburn, Calif.). Cells were washed and separated using positive selection columns (Miltenyi Biotech). The A2B5+ cell fraction was resuspended in DMEM-F12 supplemented with 1% penicillin-streptomycin, 1% glutamine (all from Invitrogen), N1 supplement (1×; Sigma), thyroid hormone T3 (2 ng/mL; Sigma) and bFGF (20 ng/mL; Sigma). PDGF (20 ng/mL; Sigma) was added to the culture media 3 days after plating. Cultures were maintained at 37° C. for 7-10 days before treatment.

### Human Adult OLGs

Tissue was obtained from surgical resections performed as treatment for nontumor-related intractable epilepsy in accordance with the guidelines net by the Biomedical Ethics Unit of McGill University. Mature OLGs were isolated as previously described. After removal of blood clots, tissue was digested with 0.25% trypsin (Invitrogen, Burlington, Canada) and 25 μg/ml of DNase I (Roche, Laval, Canada) for 30 minutes at 37° C. Cells were mechanically dissociated with a nylon mesh and separated on a linear 30% Percoll density gradient (Pharmacia Biotech) to remove myelin debris. Two overnight rounds of differential adhesion in uncoated tissue culture flasks were used to isolate the floating OLG fraction and reduce the proportion of contaminating microglia. Cells were plated in poly-L-lysine-coated glass chamber slides (Nalge Nunc International, Naperville, Ill.) in minimal essential medium with 5% fetal calf serum (Sigma), 1% penicillin-streptomycin, 1% glutamine, 0.1% glucose (all from Invitrogen), at a density of 105 cells per well. The purity of these cultures has been previously characterized.

### Antibodies and Immunocytochemistry

Primary antibodies used in this study were: mouse monoclonal anti A2B5 (CNP, Sternberger Monoclonals, Lutherville, Md.), mouse monoclonal anti-MAG (Chemicon, Temecula, Calif.), rabbit polyclonal anti-netrin-1 PN3 (Manitt, C., et al., 2001, J. Neurosci. 21:3911-3922), mouse monoclonal anti-DCC (G97-449; BD Biosciences

Pharmingen, San Jose, Calif.), rat monoclonal anti-netrin-1 (R&D Systems, Minneapolis, Minn.), rabbit polyclonal pan-Unc5h (a gift from Dr. Tony Pawson). For immunocytochemical analysis, cultures of OPCs or oligodendrocytes grown on 16-well chamber slides were fixed with 2% paraformaldehyde and incubated in blocking solution (0.3% triton X-100, 3% bovine serum albumin) for 1 hour at room temperature.

### Analysis of Oligodendrocyte Morphology and Migration Assays

OPC and OLG morphology was analyzed using the NeuronJ plugin for ImageJ (National Institutes of Health (NIH), Bethesda, Md.). The length of the longest process was measured from the base of the process to its tip. OPC migration assays were performed as described (Jarjour, A. A., et al., 2003, J. Neurosci. 23:3735-3744). Briefly, 4×104 OPCs were plated on a poly-D-lysine coated transfilter with 8 μm pores (Corning, Lowell, Mass.). The filters were placed in wells of a 12 well culture dish, and media was added to the top and bottom of the well. Netrin-1 and netrin-1ΔC were added to the bottom of the well and after 16 hrs of incubation at 370 C, cells were fixed in 4% PFA 0.1% glutaraldehyde and migrating OPCs, or Hoechst positive nuclei at the bottom of the filter, were counted.

### Biochemistry of Multiple Sclerosis and Normal Control Cases

We obtained post-mortem brain samples of cases with no CNS disease and MS from the NeuroResource tissue bank. Institute of Neurology, London, UK. The appropriate consent and ethical approvals were obtained by the tissue bank, and the studies were performed in accordance with the McGill University Health Centre research ethics board. MS plaques and normal control white matter (NCWM) samples were dissected from snap-frozen tissue blocks that had been screened with oil red O and hematoxylin staining. Plaques that were hypocellular with few or no oil red O-positive macrophages were identified as chronic. Approximately 250 mm3 of tissue was homogenized in RIPA buffer (20 mmol/L of Tris, pH 7.5, 150 mmol/L of NaCl, 1% Nonidet P-40, 0.5% sodium deoxycholate, 0.1% sodium dodecyl sulfate, 100 mmol/L of phenylmethylsulfonyl fluoride, 1 μmol/L of sodium orthovanadate, 5 μmol/L of EDTA (pH 7.4), and 1 μg/mL each of aprotinin, phenylmethylsulfonyl fluoride, leupeptin, and pepstatin), using a Caframo BOC 3030 stirrer (Caframo, Marton, Ontario, Canada) for 10 strokes at 900 rpm. SDS-PAGE and Western blotting were performed on lysed samples.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. This application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method of inhibiting tumor cell migration in a subject, the method comprising contacting a tumor cell undergoing or likely to undergo movement with a netrin polypeptide in an amount effective to decrease migration of the tumor cell, thereby modulating cell migration in the subject.

2. The method of claim 1, wherein said tumor cell is a glioblastoma cell.

3. A method as described in claim 1, wherein said netrin polypeptide is selected from the group consisting of netrin-1, VI-V netrin-1, netrin-2, netrin-3, netrin-4, netrin-G1, netrin-G2, recombinant netrin-1, recombinant netrin-2, recombinant netrin-3, recombinant-netrin-4, recombinant-netrin-G1, recombinant-netrin-G2, or modifications, variants, homologues, fragments or functional derivatives thereof.

4. A method as described in claim 1, wherein said subject is a human.

5. (canceled)

6. A method of inhibiting tumor cell migration in a subject according to claim 1, wherein the tumor cell undergoing or likely to undergo movement is contacted with a netrin polypeptide and a laminin polypeptide in an amount effective to decrease migration of the tumor cell, thereby modulating cell migration in the subject.

7-15. (canceled)

16. An isolated polypeptide comprising the sequence of the VI-V domain of netrin or a fragment, analog or modification thereof, wherein the fragment, analog or modification selectively inhibits cell growth or migration.

17. The isolated polypeptide of claim 16, wherein the netrin sequence is derived from a vertebrate netrin.

18. The isolated polypeptide of claim 17, wherein the netrin sequence is derived from human netrin.

19. The isolated polypeptide of claim 16, wherein the polypeptide is selected from the group consisting of SEQ ID NOs: 1-20, or a fragment, analog or modification thereof.

20. An isolated polynucleotide encoding the polypeptide of claim 19.

21. (canceled)

22. A pharmaceutical composition comprising the polypeptide of claim 16 and a pharmaceutically acceptable carrier.

23. A method of treating or preventing cancer in a subject in need thereof, comprising administering a therapeutically effective amount of the polypeptide of claim 16 to the subject.

24. The method of claim 23, wherein tumor cell migration is inhibited in the subject.

25. The method of claim 23, wherein the maturation of focal complexes into focal adhesions is inhibited in the subject.

26. The method of claim 23, wherein neovascularization is inhibited in the subject.

27. (canceled)

28. The method of claim 23, wherein the cancer is colorectal cancer.

29. The method of claim 23, wherein the cancer is glioblastoma.

30. The method of claim 23, wherein metastasis is inhibited in the subject.

31. A method of treating or preventing an ocular disease in a subject in need thereof, comprising administering a therapeutically effective amount of the polypeptide of claim 16 the subject.

32. The method of claim 31, wherein the disease is associated with neovascularization.

33. The method of claim 32, wherein the disease is age-related macular degeneration, diabetic retinopathy, or retinitis pigmentosa (RP).

34. The method of claim 31, wherein neovascularization is inhibited in the subject.

35. The method of claim 31, wherein cell growth, migration or branching is inhibited in the subject.

36. A method of treating or preventing unwanted neovascularization in a subject in need thereof, comprising administering a therapeutically effective amount of the polypeptide of claim 16 to the subject.

37. The method of claim 36, wherein the subject has an ocular disease or cancer.

38. The method of claim 37, wherein the disease is colorectal cancer, glioblastoma, age-related macular degeneration, diabetic retinopathy, or retinitis pigmentosa (RP).

39. (canceled)

40. (canceled)

41. (canceled)

42. A method for diagnosis or prognosis of multiple sclerosis in a subject in need thereof, comprising determining whether the VI-V domain of netrin, or a proteolytic fragment thereof, is present in CSF, in blood, or in a lesion in the subject.

43. (canceled)

44. (canceled)

45. (canceled)

46. The method of claim 1, wherein maturation of focal complexes into focal adhesions is inhibited in the subject.

47. The method of claim 1, wherein neovascularization is inhibited in the subject.

48. The method of claim 1, wherein cell growth, migration or branching is inhibited in the subject.
